(19) United States
(12) Patent Application Publication
(10) Pub. No.: US 2016/0209433 A1
(43) Pub. Date: Jul. 21, 2016

(54) MEANS AND METHODS FOR DIAGNOSING HEART FAILURE IN A SUBJECT

(71) Applicants: METANOMICS GMBH, Berlin (DE); RUPRECHT-KARLS-UNIVERSITÄT HEIDELBERG, Heidelberg (DE)

(72) Inventors: Henning Witt, Berlin (DE); Juergen Kastler, Berlin (DE); Bianca Bethan, Berlin (DE); Erik Peter, Potsdam (DE); Philipp Schatz, Berlin (DE); Hans Dirk Duengen, Berlin (DE); Tobias Daniel Trippel, Berlin (DE); Elvis Tahirovic, Berlin (DE); Hugo A. Katus, Heidelberg (DE); Norbert Frey, Kronshagen (DE); Tanja Weis, Wiesenbach (DE)

(21) Appl. No.: 14/914,815

(22) PCT Filed: Sep. 1, 2014
(86) PCT No.: PCT/EP2014/068530

§ 371 Date: Feb. 26, 2016

(30) Foreign Application Priority Data
Aug. 30, 2013 (EP) 13182441.9

(51) Int. Cl. G01N 33/92; G06F 19/28

(52) U.S. Cl. CPC G01N 33/92; G06F 19/28; G01N 2800/325; G01N 2405/08; G01N 2560/00; G01N 2570/00

(57) ABSTRACT

The present invention relates to the field of diagnostic methods. Specifically, the present invention contemplates a method for diagnosing heart failure in a subject based on a group of biomarkers. The invention also relates to tools for carrying out the aforementioned methods, such as diagnostic devices.

The present invention relates to the field of diagnostic methods. Specifically, the present invention contemplates a method for diagnosing heart failure in a subject based on a group of biomarkers and a method for monitoring progression or regression of heart failure in a subject. The invention also relates to tools for carrying out the aforementioned methods, such as diagnostic devices.

Heart failure is a severe problem in modern medicine. The impaired function of the heart can give rise to life-threatening conditions and results in discomfort for the patients suffering from heart failure. Heart failure can affect the right or the left heart, respectively, and can vary in strength. A classification system was originally developed by the New York Heart Association (NYHA). According to the classification system, the mild cases of heart failure are categorized as class I cases. These patients only show symptoms under extreme exercise. The intermediate cases show more pronounced symptoms already under less exercise (classes II and III) while class IV, shows already symptoms at rest (New York Heart Association. Diseases of the heart and blood vessels. Nomenclature and criteria for diagnosis, 6th ed. Boston: Little, Brown and co, 1964; 114).

The prevalence of heart failure steadily increases in the population of the western developed countries over the last years. One reason for said increase can be seen in an increased average life expectation due to modern medicine. The mortality rate caused by heart failure, however, could be further reduced by improved diagnostic and therapeutic approaches. The so-called “Framingham” study reported a reduction of the 5 year mortality from 70% to 59% in men and from 57% to 45% in women when comparing a time window of 1950 to 1969 with 1990 to 1999. The “Mayo” study shows a reduction from 65% to 50% for men for a time window of 1996 to 2000 compared to 1979 to 1984 and from 51% to 46% for women. Notwithstanding this reduction of the mortality rate, the overall mortality due to heart failure is still a major burden to societies. One-year mortality for NYHA class II to III patients under ACE inhibitor therapy is still between 9-12% (SOLVED) and for NYHA class IV without ACE inhibitor therapy 52% (Consensus).

Diagnostic techniques such as echocardiography are dependent on the experience of the individual investigator and, thus, not always reliable. Moreover, these techniques sometimes fail to diagnose the early onset of heart failure. Biochemical assays which are based on cardiac hormones such as Brain natriuretic peptides (BNP) are also influenced by other diseases and disorders such as renal insufficiency or depend on the overall physical condition of the patient. Nevertheless, Brain natriuretic peptides are the current gold standard for biochemically assessing heart failure. According to a recent study comparing BNP and N-terminal pro-BNP (NT-proBNP) in the diagnosis of heart failure, BNP is a better indicator for heart failure and left ventricular systolic dysfunction than NT-proBNP. In groups of symptomatic patients, a diagnostic odds ratio of 27 for BNP compares with a sensitivity of 85% and specificity of 84% in detecting heart failure (Ewald 2008, Intern Med J 38 (2):101-13.).

Recently, metabolic biomarkers for diagnosing heart failure and/or for monitoring heart failure progression or regression have been reported (see WO2011/092285 and WO2013/014286, respectively).

It is a goal of modern medicine to reliably identify and treat patients with heart failure and, in particular, to identify them at the early onset of heart failure, i.e. at the early NYHA stages I to III and in particular at NYHA stage I. Accordingly, means and methods for reliably diagnosing heart failure are still highly desired.

Therefore, the present invention relates to a method for diagnosing heart failure in a subject comprising the steps of:
a) determining in a sample of a subject suspected to suffer from heart failure the amounts of a group of biomarkers, said group comprising: Cholesterylester C18:1, Cholesterylester C18:2, a Sphingomyelin C23:0, a Sphingomyelin C24:0, and cysteine; and
b) comparing the amounts of the said biomarkers to a reference, whereby heart failure is to be diagnosed.

The method as referred to in accordance with the present invention includes a method which essentially consists of the aforementioned steps or a method which includes further steps. However, it is to be understood that the method, in a preferred embodiment, is a method carried out ex vivo, i.e. not practised on the human or animal body. The method, preferably, can be assisted by automation.

The term “diagnosing” as used herein refers to assessing whether a subject suffers from the heart failure, or not. As will be understood by those skilled in the art, such an assessment, although preferred to be, may usually not be correct for 100% of the investigated subjects. The term, however, requires that a statistically significant portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2, 0.1, or 0.05.

The term includes individual diagnosis of heart failure or its symptoms as well as continuous monitoring of a patient. Monitoring, i.e. diagnosing the presence or absence of heart failure or the symptoms accompanying it at various time points, includes monitoring of patients known to suffer from heart failure as well as monitoring of subjects known to be at risk of developing heart failure. Furthermore, monitoring can also be used to determine whether a patient is treated successfully or whether at least symptoms of heart failure can be ameliorated over time by a certain therapy. Moreover, monitoring may be used for active patient management including deciding on hospitalization, intensive care measures and/or additional qualitative monitoring as well as quantitative monitoring measures, i.e. monitoring frequency. Moreover, the term also includes classifying a subject according to the New York Heart Association (NYHA) classes for heart failure. According to this classification, heart failure can be subdivided into four classes. Subjects exhibiting class I show no limitation in activities except under strong physical exercise. Subjects exhibiting class II show slight, mild limitation of activity, while comfortable at rest or under mild exertion. Subjects exhibiting class III show marked limitation of any activity, while comfortable only at rest. Subjects exhibiting class IV show discomfort and symptoms even at rest. Preferably, heart failure to be determined in accordance with the present invention is asymptomatic heart failure, i.e. heart failure according to NYHA class I, or symptomatic heart failure, i.e. heart failure at least according to NYHA class II and/or III.

Another staging system is provided by the American Heart Association (frequently also referred to as “ACC/AHA classification”). Four stages of heart failure are subdivided: Stage A: Patients at high risk for developing HF in the future but no functional or structural heart disorder. Stage B: a structural heart disorder but no symptoms at any stage. Stage C: previous or current symptoms of heart failure in the context of an underlying structural heart problem, but managed with medical treatment. Stage D: advanced disease requiring hospital-based support, a heart transplant or palliative care. It will be understood that the method of the present invention can also be used for staging heart failure according to this system, preferably, the identified biomarkers shall allow to diagnose heart failure according to stages A to C and to discriminate between the asymptomatic stages A and B and the more severe stage C, i.e. symptomatic heart failure.

The term “heart failure” as used herein relates to an impaired function of the heart. The said impairment can be a systolic dysfunction resulting in a significantly reduced ejection fraction of blood from the heart and, thus, a reduced blood flow. Specifically, systolic heart failure is characterized by a significantly reduced left ventricular ejection fraction (LEVF), preferably, an ejection fraction of less than 50% (heart failure with reduced ejection fraction, HfrEF). Alternatively, the impairment can be a diastolic dysfunction, i.e. a failure of the ventricle to properly relax. The latter is usually accompanied by a stiffer ventricular wall. The diastolic dysfunction causes inadequate filling of the ventricle and, therefore, results in consequences for the blood flow, in general. Thus, diastolic dysfunction also results in elevated end-diastolic pressures, and the end result is comparable to the case of systolic dysfunction (pulmonary edema in left heart failure, peripheral edema in right heart failure.) Heart failure may, thus, affect the right heart (pulmonary circulation), the left heart (body circulation) or both. Techniques for measuring an impaired heart function and, thus, heart failure, are well known in the art and include echocardiography, electrophysiology, angiography, and the determination of peptide biomarkers, such as the Brain Natriuretic Peptide (BNP) or the N-terminal fragment of its propeptide, in the blood. It will be understood that the impaired function of the heart can occur permanently or only under certain stress or exercise conditions. Dependent on the strength of the symptoms, heart failure can be classified as set forth elsewhere herein. Typical symptoms of heart failure include dyspnea, chest pain, dizziness, confusion, pulmonary and/or peripheral edema. It will be understood that the occurrence of the symptoms as well as their severity may depended on the severity of heart failure and the characteristics and causes of the heart failure, systolic or diastolic or restrictive i.e. right or left heart located heart failure. Further symptoms of heart failure are well known in the art and are described in the standard text books of medicine, such as Stedman or Brunnwald.

Preferably, heart failure as used herein relates to congestive heart failure (CHF) and, more preferably, to a dilatative cardiomyopathy (DCMP), a hypertrophic cardiomyopathy (HCMP), a Heart failure with reduced ejection fraction, in general, (HFrHF) or an ischemic cardiomyopathy (ICMP). The HFrHF subgroup of heart failure patients include those suffering from DCMP and those suffering from ICMP.

Moreover, heart failure as used herein relates to symptomatic or asymptomatic heart failure.

The term “biomarker” as used herein refers to a molecular species which serves as an indicator for a disease or effect as referred to in this specification. Said molecular species can be a metabolite itself which is found in a sample of a subject. Moreover, the biomarker may also be a molecular species which is derived from said metabolite. In such a case, the actual metabolite will be chemically modified in the sample or during the determination process and, as a result of said modification, a chemically different molecular species, i.e. the analyte, will be the determined molecular species. It is to be understood that in such a case, the analyte represents the actual metabolite and has the same potential as an indicator for the respective medical condition.

A metabolite as used herein refers to at least one molecule of a specific metabolite up to a plurality of molecules of the said specific metabolite. It is to be understood further that a group of metabolites means a plurality of chemically different molecules wherein for each metabolite at least one molecule up to a plurality of molecules may be present. A metabolite in accordance with the present invention encompasses all classes of organic or inorganic chemical compounds including those being comprised by biological material such as organisms. Preferably, the metabolite in accordance with the present invention is a small molecule compound. More preferably, in case a plurality of metabolites is envisaged, said plurality of metabolites representing a metabolome, i.e. the collection of metabolites being comprised by an organism, an organ, a tissue, a body fluid or a cell at a specific time and under specific conditions.

The metabolites are small molecule compounds, such as substrates for enzymes of metabolic pathways, intermediates of such pathways or the products obtained by a metabolic pathway. Metabolic pathways are well known in the art and may vary between species. Preferably, said pathways include at least citric acid cycle, respiratory chain, glycolysis, gluconeogenesis, hexose monophosphate pathway, oxidative pentose phosphate pathway, production and β-oxidation of fatty acids, urea cycle, amino acid biosynthesis pathways, protein degradation pathways such as proteasomal degradation, amino acid degrading pathways, biosynthesis or degradation of: lipids, polyketides (including e.g. flavonoids and isoflavonoids), isoprenoids (including eg. terpenes, sterols, steroids, carotenoids, xanthophylls), carbohydrates, phenylpropanoids and derivatives, alcaloids, benzenoids, indoles, indole-sulfur compounds, porphyrines, anthocyans, hormones, vitamins, cofactors such as prosthetic groups or electron carriers, lignin, glucosinolates, purines, pyrimidines, nucleosides, nucleotides and related molecules such as tRNAs, microRNAs (miRNA) or mRNAs. Accordingly, small molecule compound metabolites are preferably composed of the following classes of compounds: alcohols, alkanes, alkenes, alkines, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives of the aforementioned compounds. The small molecules among the metabolites may be primary metabolites which are required for normal cellular function, organ function or animal growth, development or health. Moreover, small molecule metabolites further comprise secondary metabolites having essential ecological function, e.g. metabolites which allow an organism to adapt to its environment. Furthermore, metabolites are not limited to said primary and secondary metabolites and further encompass artificial small molecule compounds. Said artificial small molecule compounds are derived from exogenously provided small molecules which are administered or taken up by an organism but are not primary or secondary metabolites as defined above. For instance, artificial small molecule compounds may be metabolic products obtained from drugs by metabolic pathways of the animal. Moreover, metabolites further include peptides, oligopeptides, polypeptides, oligonucleotides and polynucleotides, such as RNA or DNA. More preferably, a metabolite has a molecular weight of 50 Da (Dalton) to 30,000 Da, most preferably less than 30,000 Da, less than 20,000 Da, less than 15,000 Da, less than 10,000 Da, less than 8,000 Da, less than 7,000 Da, less than 6,000 Da, less than 5,000 Da, less than 4,000 Da, less than 3,000 Da, less than 2,000 Da, less than 1,000 Da, less than 500 Da, less than 300 Da, less than 200 Da, less than 100 Da. Preferably, a metabolite has, however, a molecular weight of at least 50 Da. Most preferably, a metabolite in accordance with the present invention has a molecular weight of 50 Da up to 1,500 Da.

In the method according to the present invention, the aforementioned group of biomarkers is to be determined. Thus, preferably, the group of biomarkers to be determined consists of the aforementioned five biomarkers. However, the present invention, in general, envisages that one or more biomarkers in addition to Cholesterylester C18:1, Cholester-ylester C18:2, a Sphingomyelin C23:0, a Spingomyelin C24:0, and cysteine can be determined as well. Such further biomarkers encompass metabolite, protein or DNA biomarkers known in the art to be associated with heart failure or a predisposition therefor. For example, a natriuretic peptide, such as NT-proBNP, may be determined as additional biomarker.

More preferably, said at least one further biomarker is determined being selected from the biomarkers listed in Table 2. It will be understood that dependent on whether a correction, e.g., an ANOVA correction, for confounding factors is to be carried out, the at least one further biomarker can be selected from the list in Table 2 as a biomarker indicated for “without ANOVA for confounders” or indicated for “with ANOVA for confounders”.

Preferably, said “Sphingomyelin C23:0” and/or said “Sphingomyelin C24:0” referred to in accordance with the method of the invention is/are selected from the Sphingomyelins listed in Table 1B. Accordingly, a preferred Sphingomyelin C23:0 is selected from the group consisting of Sphingomyelin C23:0 (d16:1, C23:0), Sphingomyelin (d17:1,C23:0), Sphingomyelin (d18:1,C23:0), and Sphingomyelin (d18:2,C23:0), a preferred Sphingomyelin C24:0 is selected from the group consisting of Sphingomyelin (d16:1,C24:0), Sphingomyelin (d17:1,C24:0), Sphingomyelin (d18:1,C24:0), and Sphingomyelin (d18:2,C24:0).

The term “sample” as used herein refers to samples from body fluids, preferably, blood, plasma, serum, saliva or urine, or samples derived, e.g., by biopsy, from cells, tissues or organs, in particular from the heart. More preferably, the sample is a blood, plasma or serum sample, most preferably, a plasma sample. Biological samples can be derived from a subject as specified elsewhere herein. Techniques for obtaining the aforementioned different types of biological samples are well known in the art. For example, blood samples may be obtained by blood taking while tissue or organ samples are to be obtained, e.g., by biopsy.

The aforementioned samples are, preferably, pre-treated before they are used for the method of the present invention. As described in more detail below, said pre-treatment may include treatments required to release or separate the compounds or to remove excessive material or waste. Suitable techniques comprise centrifugation, extraction, fractioning, ultrafiltration, protein precipitation followed by filtration and purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

The term “subject” as used herein relates to animals and, preferably, to mammals. More preferably, the subject is a primate and, most preferably, a human. The subject, preferably, is suspected to suffer from heart failure, more preferably, it may already show some or all of the symptoms associated with the disease. However, also encompassed as subjects suspected to suffer from heart failure are those, which belong into risk groups or subjects that are included in disease screening projects or measures. More preferably, the subject is an asymptomatic subject exhibiting symptoms according to NYHA class I or a symptomatic subject exhibiting symptoms according to NYHA class II and/or III. Moreover, the subject shall also preferably exhibit congestive systolic heart failure due to contractile dysfunction such as dilated cardiomyopathy. Preferably, the subject, however, is besides the aforementioned diseases and disorders apparently healthy. In particular, it shall, preferably, not exhibit symptoms according to NYHA class IV patients or suffer from apoplex (stroke), myocardial infarction within the last 4 month before the sample has been taken or from acute or chronic inflammatory diseases and malignant tumors. Furthermore, the subject is preferably in stable medications within the last 4 weeks before the sample was taken.

Preferably, the subject is an adult. Also preferably, the subject is older than 40 years of age, or older than 50 years of age. The subject to be tested preferably may have a history of myocardial infarction and/or may suffer from diabetes type II. Moreover, the subject preferably may suffer from hypertension.

The term “determining the amount” as used herein refers to determining at least one characteristic feature of a biomarker to be determined by the method of the present invention in the sample. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemiluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the said at least one biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z information, as well as an intensity value being related to the abundance of the said biomarker (i.e. its amount) in the sample.

As discussed before, each biomarker comprised by a sample may be, preferably, determined in accordance with the present invention quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present is enlarged or diminished with respect to a second sample comprising said biomarker in a second amount. In a preferred embodiment said second sample comprising said biomarker shall be a calculated reference as specified elsewhere herein. Quantitatively analysing a biomarker, thus, also includes what is sometimes referred to as semi-quantitative analysis of a biomarker.

Moreover, determining as used in the method of the present invention, preferably, includes using a compound separation step prior to the analysis step referred to before. Preferably, said compound separation step yields a time resolved separation of the metabolites comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or GC are chromatographic techniques to be envisaged by the method of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). Most preferably, LC-MS and/or GC-MS are used as described in detail below. Said techniques are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference. As an alternative or in addition to mass spectrometry techniques, the following techniques may be used for compound determination: nuclear magnetic resonance (NMR), magnetic resonance imaging (MRI), Fourier transform infrared analysis (FTIR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado. The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

Moreover, the at least one biomarker can also be determined by a specific chemical or biological assay. Said assay shall comprise means which allow to specifically detect the at least one biomarker in the sample. Preferably, said means are capable of specifically recognizing the chemical structure of the biomarker or are capable of specifically identifying the biomarker based on its capability to react with other compounds or its capability to elicit a response in a biological read out system (e.g., induction of a reporter gene). Means which are capable of specifically recognizing the chemical structure of a biomarker are, preferably, antibodies or other proteins which specifically interact with chemical structures, such as receptors or enzymes. Specific antibodies, for instance, may be obtained using the biomarker as antigen by methods well known in the art. Antibodies as referred to herein include both polyclonal and monoclonal antibodies, as well as fragments thereof, such as Fv, Fab and F(ab)₂ fragments that are capable of binding the antigen or hapten. The present invention also includes humanized hybrid antibodies wherein amino acid sequences of a non-human donor antibody exhibiting a desired antigen-specificity are combined with sequences of a human acceptor antibody. Moreover, encompassed are single chain antibodies. The donor sequences will usually include at least the antigen-binding amino acid residues of the donor but may comprise other structurally and/or functionally relevant amino acid residues of the donor antibody as well. Such hybrids can be prepared by several methods well known in the art. Suitable proteins which are capable of specifically recognizing the biomarker are, preferably, enzymes which are involved in the metabolic conversion of the said biomarker. Said enzymes may either use the biomarker as a substrate or may convert a substrate into the biomarker. Moreover, said antibodies may be used as a basis to generate oligopeptides which specifically recognize the biomarker. These oligopeptides shall, for example, comprise the enzyme's binding domains or pockets for the said biomarker. Suitable antibody and/or enzyme based assays may be RIA (radioimmunoassay), ELISA (enzyme-linked immunosorbent assay), sandwich enzyme immune tests, electrochemiluminescence sandwich immunoassays (ECLIA), dissociation-enhanced lanthanide fluoro immuno assay (DELFIA) or solid phase immune tests. Moreover, the biomarker may also be determined based on its capability to react with other compounds, i.e. by a specific chemical reaction. Further, the biomarker may be determined in a sample due to its capability to elicit a response in a biological read out system. The biological response shall be detected as read out indicating the presence and/or the amount of the biomarker comprised by the sample. The biological response may be, e.g., the induction of gene expression or a phenotypic response of a cell or an organism. In a preferred embodiment the determination of the least one biomarker is a quantitative process, e.g., allowing also the determination of the amount of the at least one biomarker in the sample.

As described above, said determining of the at least one biomarker can, preferably, comprise mass spectrometry (MS). Mass spectrometry as used herein encompasses all techniques which allow for the determination of the molecular weight (i.e. the mass) or a mass variable corresponding to a compound, i.e. a biomarker, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. How to apply these techniques is well known to the person skilled in the art. Moreover, suitable devices are commercially available. More preferably, mass spectrometry as used herein relates to LC-MS and/or GCMS, i.e. to mass spectrometry being operatively linked to a prior chromatographic separation step. More preferably, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) fragmentation of the ion selected in step a) by applying an acceleration voltage in an additional subsequent quadrupole which is filled with a collision gas and acts as a collision chamber, c) selection of a mass/charge quotient of an ion created by the fragmentation process in step b) in an additional subsequent quadrupole, whereby steps a) to c) of the method are carried out at least once and analysis of the mass/charge quotient of all the ions present in the mixture of substances as a result of the ionisation process, whereby the quadrupole is filled with collision gas but no acceleration voltage is applied during the analysis. Details on said most preferred mass spectrometry to be used in accordance with the present invention can be found in WO2003/073464.

The mass spectrometry preferably comprises an ionization step in which the biomarkers to be determined are ionized. Ionization of the biomarkers can be carried out by any method deemed appropriate, in particular by electron impact ionization, fast atom bombardment, electrospray ionization (ESI), atmospheric pressure chemical ionization (APOI), matrix assisted laser desorption ionization (MALDI).

In a preferred embodiment the ionization is carried out as described in the Examples section.

More preferably, said mass spectrometry is liquid chromatography (LC) MS and/or gas chromatography (GC) MS. Liquid chromatography as used herein refers to all techniques which allow for separation of compounds (i.e. metabolites) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. Gas chromatography as applied in accordance with the present invention, in principle, operates comparable to liquid chromatography. However, rather than having the compounds (i.e. metabolites) in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time which is required for passing through the column. Moreover, in the case of gas chromatography it is preferably envisaged that the compounds are derivatised prior to gas chromatography. Suitable techniques for derivatisation are well known in the art. Preferably, derivatisation in accordance with the present invention relates to methoxymation and trimethylsilylation of, preferably, polar compounds and transmethylation, methoxymation and trimethylsilylation of, preferably, non-polar (i.e. lipophilic) compounds.

The term “reference” refers to values of characteristic features of each of the biomarker which can be correlated to a medical condition, i.e. the presence or absence of the disease, diseases status or an effect referred to herein. Preferably, a reference is a threshold value (e.g., an amount or ratio of amounts) for a biomarker whereby values found in a sample to be investigated which are higher than or essentially identical to the threshold are indicative for the presence of a medical condition while those being lower are indicative for the absence of the medical condition. It will be understood that also preferably, a reference may be a threshold value for a biomarker whereby values found in a sample to be investigated which are lower or identical than the threshold are indicative for the presence of a medical condition while those being higher are indicative for the absence of the medical condition.

In accordance with the aforementioned method of the present invention, a reference is, preferably, a reference obtained from a sample from a subject or group of subjects known to suffer from heart failure. In such a case, a value for each of the biomarkers of the group found in the test sample being essentially identical is indicative for the presence of the disease. Moreover, the reference, also preferably, could be from a subject or group of subjects known not to suffer from heart failure, preferably, an apparently healthy subject. In such a case, a value for each of the biomarkers of the group found in the test sample being altered with respect to the reference is indicative for the presence of the disease. The same applies mutatis mutandis for a calculated reference, most preferably the average or median, for the relative or absolute value of the at least one biomarker of a population of individuals comprising the subject to be investigated. The absolute or relative values of the biomarkers of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 or 10,000 subjects. It is to be understood that the subject to be diagnosed by the method of the present invention and the subjects of the said plurality of subjects are of the same species.

The value for a biomarker of the test sample and the reference values are essentially identical, if the values for the characteristic features and, in the case of quantitative determination, the intensity values are essentially identical. Essentially identical means that the difference between two values is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value, preferably, the 50^{th}, 60^{th}, 70^{th}, 80^{th}, 90^{th} or 95^{th} percentile of the reference value. Statistical test for determining whether two amounts are essentially identical are well known in the art and are also described elsewhere herein.

In a preferred embodiment the value for the characteristic feature can also be a calculated output such as score of a classification algorithm like “elastic net” as set forth elsewhere herein.

An observed difference for two values, on the other hand, shall be statistically significant. A difference in the relative or absolute value is, preferably, significant outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference value. Preferred changes and ratios of the medians are described in the accompanying Tables as well as in the Examples.

Preferably, the reference, i.e. values for at least one characteristic feature of the biomarkers or ratios thereof, will be stored in a suitable data storage medium such as a database and are, thus, also available for future assessments.

The term “comparing”, preferably, refers to determining whether the determined value of the biomarkers is essentially identical to a reference or differs therefrom. Preferably, a value for a biomarker is deemed to differ from a reference if the observed difference is statistically significant which can be determined by statistical techniques referred to elsewhere in this description. If the difference is not statistically significant, the biomarker value and the reference are essentially identical. Based on the comparison referred to above, a subject can be assessed to suffer from the disease, or not.

For the specific biomarkers referred to in this specification, preferred values for the changes in the relative amounts or ratios (i.e. the changes expressed as the ratios of the means) or the kind of regulation (i.e. “up”- or “down”-regulation or increase or decrease resulting in a higher or lower relative and/or absolute amount or ratio) are indicated in the Tables 1A, 1B or 2 and in the Examples below. The ratio of means indicates the degree of increase or decrease, e.g., a value of 2 means that the amount is twice the amount of the biomarker compared to the reference. Moreover, it is apparent whether there is an “up- regulation” or a “down-regulation”. In the case of an “up-regulation” the ratio of the mean shall exceed 1.0 while it will be below 1.0 in case of a “down”-regulation. Accordingly, the direction of regulation can be derived from the Tables as well. It will be understood that instead of the means, medians could be used as well.

Preferably, the values or ratios determined in a sample of a subject according to the present invention are adjusted for age, BMI, gender or other existing diseases, e.g., the presence or absence of diabetes before being comparing to a reference. Alternatively, the references can be derived from values or ratios which have likewise been adjusted for age, BMI, gender or other confounders, such as diseases, e.g., the presence or absence of diabetes. Such an adjustment can be made by deriving the references and the underlying values or ratios from a group of subjects the individual subjects of which are essentially identical with respect o theses parameters to the subject to be investigated. Alternatively, the adjustment may be done by statistical calculations. Thus, a correction for confounders may be carried out. However, as set forth elsewhere herein, a correction for confounders may not be carried out.

The comparison is, preferably, assisted by automation. For example, a suitable computer program comprising algorithms for the comparison of two different data sets (e.g., data sets comprising the values of the characteristic feature(s)) may be used. Such computer programs and algorithms are well known in the art. Notwithstanding the above, a comparison can also be carried out manually.

In the context of step b) of the present invention, the amounts of a group of biomarkers as referred to in step a) of the methods of the present invention shall be compared to references for the individual biomarkers. Thereby, the presence or absence of a disease as referred to herein is diagnosed.

In particular, it is also envisaged to calculate a score based on the amounts of the individual biomarkers, i.e. a single score, and to compare this score to a reference score. The calculated score combines information on the amounts of the group of biomarkers. The score can be regarded as a classifier parameter for diagnosing heart failure. In particular, it enables the person who provides the diagnosis based on a single score. Thus, the person does not have to interpret the entire information on the amounts of the individual biomarkers.

Thus, in a preferred embodiment of the present invention, the comparison of the amounts to a reference as set forth in step b) of the method of the present invention encompasses step b1) of calculating a score based on the determined amounts of the biomarkers as referred to in step a), and step b2) of comparing the, thus, calculated score to a reference score.

Thus, the present invention, in particular, a method for diagnosing heart failure in a subject comprising the steps of:
a) determining in a sample of a subject suspected to suffer from heart failure the amounts of a group of biomarkers, said group comprising: Cholesterylester C18:1, Cholesterylester C18:2, a Sphingomyelin C23:0, a Spingomyelin C24:0, and cysteine; and
b1) calculating a score based on the determined amounts of the biomarkers as referred to in step a), and
b2) comparing the, thus, calculated score to a reference score, whereby heart failure is to be diagnosed.

Preferably, the reference score shall allow for differentiating whether a subject suffers from a disease as referred to herein, or not. Preferably, the diagnosis is made by assessing whether the score of the test subject is above or below the reference score. It is not necessary to provide an exact reference score. A relevant reference score can be obtained by correlating the sensitivity and specificity and the sensitivity/specificity for any score. A reference score resulting in a high sensitivity results in a lower specificity and vice versa.

Preferably, the score is calculated based on a suitable scoring algorithm. Said scoring algorithm, preferably, shall allow for differentiating whether a subject suffers from a disease as referred to herein, or not, based on the amounts of the biomarkers to be determined.

Preferably, said scoring algorithm has been previously determined by comparing the information regarding the amounts of the individual biomarkers as referred to in step a) in samples from patients suffering from a disease as referred to herein and from patients not suffering from said disease. Accordingly, step b) may also comprise step b0) of determining or implementing a scoring algorithm. Preferably, this step is carried out prior steps b1) and b2).

A suitable scoring algorithm can determined with the group of biomarkers referred to in step a) by the skilled person without further ado. E.g., the scoring algorithm may be a mathematical function that uses information regarding the amounts of the biomarkers in a cohort of subjects suffering from heart failure and not suffering from heart failure. Methods for determining a scoring algorithm are well know in the art and including Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naïve Bayes, Naïve Bayes Simple, Naïve Bayes Up, IB1, Ibk, Kstar, LWL, AdaBoost, ClassViaRegression, Decorate, Multiclass Classifier, Random Committee, j48, LMT, NBTree, Part, Random Forest, Ordinal Classifier, Sparse Linear Programming (SPLP), Sparse Logistic Regression (SPLR), Elastic net, Support Vector Machine, Prediction of Residual Error Sum of Squares (PRESS), Penalized Logistic Regression, Mutual Information. Preferably, the scoring algorithm is determined with or without correction for confounders as set forth elsewhere herein.

Preferably, the scoring algorithm is determined with an elastic net with the group of biomarkers (see also Examples section).

Advantageously, it has been found in the study underlying the present invention that the amounts of the group of specific biomarkers referred to above are indicators for heart failure. Accordingly, the group of biomarkers as specified above in a sample can, in principle, be used for assessing whether a subject suffers from heart failure. This is particularly helpful for an efficient diagnosis of the disease as well as for improving of the pre-clinical and clinical management of heart failure as well as an efficient monitoring of patients. Moreover, the findings underlying the present invention will also facilitate the development of efficient drug-based therapies or other interventions including nutritional diets against heart failure as set forth in detail below.

The definitions and explanations of the terms made above apply mutatis mutandis for the following embodiments of the present invention except specified otherwise herein below.

In accordance with the studies underlying the present invention, particular groups of biomarkers comprising one or more biomarkers in addition to the aforementioned group have been identified as suitable biomarkers for identifying subjects suffering from congestive heart failure (CHF) and, more preferably, a dilatative cardiomyopathy (DCMP), a hypertrophic cardiomyopathy (HCMP), a Heart failure with reduced ejection fraction, in general, (HFrHF) or an ischemic cardiomyopathy (ICMP).

Thus, in a preferred embodiment of the method of the present invention, said heart failure is congestive heart failure according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), TAG_Stearic acid (C18:0), Normetanephrine, and Mannose provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Normetanephrine, TAG_Stearic acid (C18:0), Noradrenaline (Norepinephrine), and Behenic acid (C22:0) provided that a correction for confounders is carried out.

In another preferred embodiment of the method of the present invention said heart failure is congestive heart failure according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Mannose, Normetanephrine, Lignoceric acid (C24:0), and TAG_Stearic acid (C18:0) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Mannose, Noradrenaline (Norepinephrine), Lignoceric acid (C24:0), and TAG_Stearic acid (C18:0) provided that a correction for confounders is carried out.

In a further preferred embodiment of the method of the present invention, said heart failure is DCMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, TAG_Stearic acid (C18:0), Noradrenaline (Norepinephrine), alpha-Ketoglutarate, trans-4-Hydroxyproline, and Uric acid provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), TAG_Stearic acid (C18:0), alpha-Ketoglutarate, Uric acid, and Lignoceric acid (C24:0) provided that a correction for confounders is carried out.

In a preferred embodiment of the method of the present invention said heart failure is DCMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Uric acid, Lignoceric acid (C24:0), TAG_Stearic acid (C18:0), and Noradrenaline (Norepinephrine) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Uric acid, TAG_Stearic acid (C18:0), and Mannose provided that a correction for confounders is carried out.

In a preferred embodiment of the method of the present invention said heart failure is HCMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, TAG_Stearic acid (C18:0), Mannose, Pyruvate, and Uric acid provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, TAG_Stearic acid (C18:0), Pyruvate, Taurine, Uric acid, and Mannose provided that a correction for confounders is carried out.

In a preferred embodiment of the method of the present invention said heart failure is HCMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, Cystine, Lactate, Lignoceric acid (C24:0), alpha-Ketoglutarate, and Mannose provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Lactate, Lignoceric acid (C24:0), TAG_Stearic acid (C18:0), Cystine, and alpha-Ketoglutarate provided that a correction for confounders is carried out.

In a preferred embodiment of the method of the present invention said heart failure is HFrEF according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), TAG_Stearic acid (C18:0), Normetanephrine, and Behenic acid (C22:0) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Normetanephrine, TAG_Stearic acid (C18:0), and Behenic acid (C22:0) provided that a correction for confounders is carried out.

In a preferred embodiment of the method of the present invention said heart failure is HFrEF according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Mannose, Glycine, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, TAG_Stearic acid (C18:0), and Behenic acid (C22:0) provided that a correction for confounders is carried out.

In a preferred embodiment of the method of the present invention said heart failure is ICMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that a correction for confounders is carried out.

In a preferred embodiment of the method of the present invention said heart failure is ICMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, Behenic acid (C22:0), and Normetanephrine provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Behenic acid (C22:0), Mannose, and TAG_Stearic acid (C18:0) provided that a correction for confounders is carried out.

The present invention also relates to a method for identifying whether a subject is in need for a therapy of heart failure or a change of therapy comprising the steps of the methods of the present invention and the further step of identifying a subject in need if heart failure is diagnosed.

The phrase “in need for a therapy of heart failure” as used herein means that the disease in the subject is in a status where therapeutic intervention is necessary or beneficial in order to ameliorate or treat heart failure or the symptoms associated therewith. Accordingly, the findings of the studies underlying the present invention do not only allow diagnosing heart failure in a subject but also allow for identifying subjects which should be treated by a heart failure therapy or whose heart failure therapy needs adjustment. Once the subject has been identified, the method may further include a step of making recommendations for a therapy of heart failure.

A therapy of heart failure as used in accordance with the present invention, preferably, relates to a therapy which comprises or consists of the administration of at least one drug selected from the group consisting of: ACE Inhibitors (ACEI), Beta Blockers, AT1-Inhibitors, Aldosteron Antagonists, Renin Antagonists, Diuretics, Ca-Sensitizer, Digitalis Glykosides, antiplatelet agents, Vitamin-K-Antagonists, polypeptides of the protein S100 family (as disclosed by DE000003922873A1, DE000019815128A1 or DE000019915485A1 hereby incorporated by reference), natriuretic peptides such as BNP (Nesiritide (human recombinant Brain Natriuretic Peptide—BNP)) or ANP.

As a rule, patients are preferably treated with medication as recommended by the guidelines of the European Society of Cardiology (Ref: European Heart Journal (2012), 33:1787-1847).

Preferably, the therapy comprises the administration of Diuretics, Aldosteron Antagonists and/or ACE Inhibitors, if the heart failure is DCMP.

Preferably, the therapy comprises the administration of Diuretics, Aldosteron Antagonists and/or ACE Inhibitors, if the heart failure is ICMP. Also preferred are Vitamin-K-antagonists and antiplatelet agents.

Preferably, the therapy comprises the administration of Vitamin-K-Antagonists and/or antiplatelet agents), if the heart failure is HCMP.

The present invention further relates to a method for determining whether a therapy against heart failure is successful in a subject comprising the steps of the methods of the present invention and the further step of determining whether a therapy is successful if no heart failure is diagnosed.

It is to be understood that a heart failure therapy will be successful if heart failure or at least some symptoms thereof can be treated or ameliorated compared to an untreated subject. Moreover, a therapy is also successful as meant herein if the disease progression can be prevented or at least slowed down compared to an untreated subject.

The invention also relates to a method for establishing an aid for diagnosing whether a subject suffering from heart failure, or not, is contemplated, said method comprising:

a) selecting a subject suspected to suffer from heart failure;

b) obtaining a sample from said subject to be tested;

c) pretreating said sample in preparation for analysis;

d) determining the amount of each of the biomarkers of the group of biomarkers of as referred to above in a sample of said subject, said determining comprises (i) bringing the sample into contact with a detection agent that specifically binds to said at least one biomarker for a time sufficient to allow for the formation of a complex of the said detection agent and the biomarker from the sample, (ii) measuring the amount of the formed complex, wherein the said amount of the formed complex is proportional to the amount of biomarker present in the sample, and (iii) transforming the amount of the formed complex into an amount of biomarker reflecting the amount of the biomarker present in the sample;

e) comparing said amount to a reference; and

f) establishing an aid for diagnosing heart failure based on the result of the comparison made in step e).

A suitable detection agent may be, preferably, an antibody which is specifically binds to the at least one biomarker in a sample of a subject to be investigated by the method of the invention.

Another detection agent that can be applied, preferably, may be an aptamere which specifically binds to at least one biomarker in the sample. In yet a preferred embodiment, the sample is removed from the complex formed between the detection agent and the at least one biomarker prior to the measurement of the amount of formed complex. Accordingly, in a preferred embodiment, the detection agent may be immobilized on a solid support. In yet a preferred embodiment, the sample can be removed from the formed complex on the solid support by applying a washing solution. The formed complex shall be proportional to the amount of the at least one biomarker present in the sample. It will be understood that the specificity and/or sensitivity of the detection agent to be applied defines the degree of proportion of at least one biomarker comprised in the sample which is capable of being specifically bound. Further details on how the determination can be carried out are also found elsewhere herein. The amount of formed complex shall be transformed into an amount of at least one biomarker reflecting the amount indeed present in the sample. Such an amount, preferably, may be essentially the amount present in the sample or may be, preferably, an amount which is a certain proportion thereof due to the relationship between the formed complex and the amount present in the original sample.

In yet a preferred embodiment of the aforementioned method, step d) may be carried out by an analyzing unit, in an aspect, an analyzing unit as defined elsewhere herein.

In a preferred embodiment of the method of the invention, the amount determined in step d) is compared to a reference. Preferably, the reference is a reference as defined elsewhere herein. In yet another preferred embodiment, the reference takes into account the proportional relationship between the measured amount of complex and the amount present in the original sample. Thus, the references applied in a preferred embodiment of the method of the invention are artificial references which are adopted to reflect the limitations of the detection agent that has been used. In another preferred embodiment, said relationship can be also taken into account when carrying out the comparison, e.g., by including a normalization and/or correction calculation step for the determined amount prior to actually comparing the value of the determined amount and the reference. Again, the normalization and/or correction calculation step for the determined amount adopts the comparison step such that the limitations of the detection agent that has been used are reflected properly. Preferably, the comparison is carried out automatically, e.g., assisted by a computer system or the like.

The aid for diagnosing is established based on the comparison carried out in step b) by allocating the subject either into a group of subjects suffering from heart failure with certain likelihood or a group of subjects not suffering therefrom. As discussed elsewhere herein already, the allocation of the investigated subject must not be correct in 100% of the investigated cases. Moreover, the groups of subjects into which the investigated subject is allocated are artificial groups in that they are established based on statistical considerations, i.e. a certain preselected degree of likelihood based on which the method of the invention shall operate. Thus, the method may establish an aid of diagnosis which may, in an aspect, require further strengthening of the diagnosis by other techniques. Preferably, the aid for diagnosing is established automatically, e.g., assisted by a computer system or the like.

In a preferred embodiment of the method of the invention, the determination of the at least one biomarker is achieved by mass spectroscopy techniques (preferably GCMS and/or LCMS), NMR or others referred to herein above. In such cases, preferably, the sample to be analyzed is pretreated. Said pretreatment, preferably, includes obtaining of the at least one biomarker from sample material, e.g., plasma or serum may be obtained from whole blood or the at least one biomarker may even be specifically extracted from sample material. Moreover, for GCMS, further sample pretreatment such as derivatization of the at least one biomarker is, preferably, required. In a preferred embodiment, the derivatization is carried out as described in the Examples section. Furthermore, pretreatment also, preferably, includes diluting sample material and adjusting or normalizing the concentration of the components comprised therein. To this end, preferably, normalization standards may be added to the sample in predefined amounts which allow for making a comparison of the amount of the at least one biomarker and the reference and/or between different samples to be analyzed.

In another preferred embodiment of the method of the invention, the determination of the biomarkers of the biomarkers as referred to above in a sample of a subject, is achieved by applying at least two different techniques of determination, wherein certain of said biomarkers are determined with a first technique and certain others of said biomarkers are determined with a second technique etc. For example certain of said biomarkers are determined via a mass spectrometry technique and certain others of said biomarkers are determined via an antibody test or an enzymatic based test. It is also possible to determine certain of said biomarkers via a combination of liquid chromatography and mass spectrometry and and certain others of said biomarkers are determined via a combination of gas chromatography and mass spectrometry.

The method of the present invention, in a preferred embodiment, furthermore further comprises a step of recommending and/or managing the subject according to the result of the aid of diagnosis established in step c). Such a recommendation may, in an aspect, be an adaptation of life style, nutrition and the like aiming to improve the life circumstances, the application of therapeutic measures as set forth elsewhere herein in detail, and/or a regular disease monitoring.

In another preferred embodiment of the aforementioned method, steps e) and/or f) are carried out by an evaluation unit as set forth elsewhere herein.

The method, in another preferred embodiment, also includes a step of managing or treating a subject according to the recommendation or diagnostic result. Preferably, said treating encompasses administering to the subject a therapeutically effective dose of at least one drug selected from the group consisting of: ACE Inhibitors (ACEI), Beta Blockers, AT1-Inhibitors, Aldosteron Antagonists, Renin Antagonists, Diuretics, Ca-Sensitizer, Digitalis Glykosides, antiplatelet agents, Vitamin-K-Antagonists, polypeptides of the protein S100 family (as disclosed by DE000003922873A1, DE000019815128A1 or DE000019915485A1 hereby incorporated by reference), natriuretic peptides such as BNP (Nesiritide (human recombinant Brain Natriuretic Peptide—BNP)) or ANP.

Further, the present invention also in an aspect pertains to a method of treating heart failure comprising the steps of the method for identifying whether a subject is in need for a therapy of heart failure or a change of therapy comprising the steps of the methods of the present invention, the further step of identifying a subject in need if heart failure is diagnosed and the further step of treating the subject accordingly.

The definitions and explanations of the terms made above apply mutatis mutandis for the following embodiments of the present invention except specified otherwise herein below.

The present invention also relates to a method of monitoring progression or regression of heart failure in a subject comprising the steps of:

a) determining in a first and a second sample of said subject the amounts of a group of biomarkers, said group comprising: Cholesterylester C18:1, Cholesterylester C18:2, a Sphingomyelin C23:0, a Spingomyelin C24:0, and cysteine, wherein said first sample has been obtained prior to said second sample; and

b) comparing the amounts determined in the first sample with the amounts determined in the second sample, whereby progression or regression of heart failure is to be diagnosed.

As set forth elsewhere herein, it is also envisaged to determine in the first and the second sample the amounts of further biomarkers known in the art to be associated with heart failure or a predisposition therefor. For example, a natriuretic peptide, such as NT-proBNP, may be determined as additional biomarker.

More preferably, said further biomarker is a biomarker selected from the biomarkers listed in Table 2.

Preferably, said “Spingomyelin C23:0” and/or said “Spingomyelin C24:0” referred to in accordance with the method of the invention is/are selected from the Sphingomyelins listed in Table 1B.

In particular, the present invention envisages a method of monitoring progression or regression of heart failure in a subject comprising the steps of:

a) determining in a first and a second sample of said subject the amounts of a group of biomarkers, wherein said first sample has been obtained prior to said second sample; and

b) comparing the amounts determined in the first sample with the amounts determined in the second sample, whereby progression or regression of heart failure is to be diagnosed, wherein said heart failure is HCMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, TAG_Stearic acid (C18:0), Mannose, Pyruvate, and Uric acid provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, TAG_Stearic acid (C18:0), Pyruvate, Taurine, Uric acid, and Mannose provided that a correction for confounders is carried out,

wherein said heart failure is HCMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, Cystine, Lactate, Lignoceric acid (C24:0), alpha-Ketoglutarate, and Mannose provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Lactate, Lignoceric acid (C24:0), TAG_Stearic acid (C18:0), Cystine, and alpha-Ketoglutarate provided that a correction for confounders is carried out,

wherein said heart failure is HFrEF according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), TAG_Stearic acid (C18:0), Normetanephrine, and Behenic acid (C22:0) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Normetanephrine, TAG_Stearic acid (C18:0), and Behenic acid (C22:0) provided that a correction for confounders is carried out,

wherein said heart failure is HFrEF according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Mannose, Glycine, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, TAG_Stearic acid (C18:0), and Behenic acid (C22:0) provided that a correction for confounders is carried out.

wherein said heart failure is ICMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that a correction for confounders is carried out,

wherein said heart failure is ICMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, Behenic acid (C22:0), and Normetanephrine provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Behenic acid (C22:0), Mannose, and TAG_Stearic acid (C18:0) provided that a correction for confounders is carried out.

The term “monitoring” as used herein refers to determining heart failure progression or heart failure regression between the time point when the first sample has been taken until the time point when the second sample has been taken. Monitoring can also be used to determine whether a patient is treated successfully or whether at least symptoms of heart failure can be ameliorated over time by a certain therapy.

The term “progression” as used herein refers to the worsening of heart failure or its accompanying symptoms. Likewise, the term “regression” as used herein refers to an amelioration of heart failure or its accompanying syndromes. It will be understood that a regression of heart failure, preferably, occurs after application of a therapy of heart failure as specified elsewhere herein. Accordingly, the aforementioned method can be, preferably, also applied in order to determining whether a therapy against heart failure is successful in a subject.

As set forth herein above, the first sample shall have been obtained prior to the second sample. Preferably, the first sample has been obtained at least one month, more preferably, at least three months, even more preferably at least six months, and most preferably, at least nine months prior to the second sample.

For the specific biomarkers referred to in this specification, the kind of regulation (i.e. “up”- or “down”-regulation or increase or decrease resulting in a higher or lower relative and/or absolute amount or ratio) are indicated in the Tables 1A, 1B or 2 and in the Examples below. If the biomarker is marked as “upregulated” in the tables (“up”), an increase of the amount of the biomarker in the second sample as compared to the first sample is indicative for a progression of heart failure, whereas a decreased amount of the biomarker in the second sample as compared to the first sample is indicative for a regression of heart failure. If the biomarker marked as “downregulated” in the tables (“down”), a decrease of the amount of the biomarker in the second sample as compared to the first sample is indicative for a progression of heart failure, whereas an increased amount of the biomarker in the second sample as compared to the first sample is indicative for a regression of heart failure. Preferably, the increase or decrease is statistically significant.

It is also envisaged to calculate a score based on the increases or decreases of the amounts (in the second sample as compared to the first sample) of the individual biomarkers and to compare this score to a reference score. The, thus, calculated score combines information on the increases or decreases of the amounts of the group of biomarkers. The score can be regarded as a classifier parameter for monitoring heart failure.

Thus, in a preferred embodiment of the aforementioned method, the method further comprises the step of calculating a score based on the determined increases and/or decreases of the amounts of the biomarkers as referred to in step a). Based on this score, heart failure can be monitored.

The aforementioned methods for the determination of the group of biomarkers can be implemented into a device. A device as used herein shall comprise at least the aforementioned means. Moreover, the device, preferably, further comprises means for comparison and evaluation of the detected characteristic feature(s) of the at least one biomarker and, also preferably, the determined signal intensity. The means of the device are, preferably, operatively linked to each other. How to link the means in an operating manner will depend on the type of means included into the device. For example, where means for automatically qualitatively or quantitatively determining the biomarker are applied, the data obtained by said automatically operating means can be processed by, e.g., a computer program in order to facilitate the assessment. Preferably, the means are comprised by a single device in such a case. Said device may accordingly include an analyzing unit for the biomarker and a computer unit for processing the resulting data for the assessment. Preferred devices are those which can be applied without the particular knowledge of a specialized clinician, e.g., electronic devices which merely require loading with a sample.

Alternatively, the methods for the determination of the at least one biomarker can be implemented into a system comprising several devices which are, preferably, operatively linked to each other. Specifically, the means must be linked in a manner as to allow carrying out the method of the present invention as described in detail above. Therefore, operatively linked, as used herein, preferably, means functionally linked. Depending on the means to be used for the system of the present invention, said means may be functionally linked by connecting each mean with the other by means which allow data transport in between said means, e.g., glass fiber cables, and other cables for high throughput data transport. Nevertheless, wireless data transfer between the means is also envisaged by the present invention, e.g., via LAN (Wireless LAN, W-LAN). A preferred system comprises means for determining biomarkers. Means for determining biomarkers as used herein encompass means for separating biomarkers, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS and/or GC-MS are used in the system of the present invention as described in detail elsewhere in the specification. Further comprised shall be means for comparing and/or analyzing the results obtained from the means for determination of biomarkers. The means for comparing and/or analyzing the results may comprise at least one databases and an implemented computer program for comparison of the results. Preferred embodiments of the aforementioned systems and devices are also described in detail below.

Therefore, the present invention relates to a diagnostic device comprising:

a) an analysing unit comprising one at least one detector for a group of biomarkers, said group comprising Cholesterylester C18:1, Cholesterylester C18:2, a Sphingomyelin C23:0, a Spingomyelin C24:0, and cysteine, wherein said analyzing unit is adapted for determining the amounts of the said biomarkers detected by the at least one detector, and, operatively linked thereto;

b) an evaluation unit comprising a computer comprising tangibly embedded a computer program code for carrying out a comparison of the determined amounts of the group of biomarkers and reference amounts and a data base comprising said reference amounts for the said biomarkers whereby it will be diagnosed whether a subject suffers from heart failure.

Preferably, the computer program code is capable of executing step of the method of the present invention as specified elsewhere herein in detail. Accordingly, the device can be used for diagnosing heart failure as specified herein based on a sample of a subject.

In a preferred embodiment, the device comprises a further database comprising the kind of regulation and/or fold of regulation values indicated for the respective biomarkers in any one of Tables 1A, 1B or 2 and a further tangibly embedded computer program code for carrying out a comparison between the determined kind of regulation and/or fold of regulation values and those comprised by the database.

In a preferred embodiment of the device of the present invention, said heart failure is congestive heart failure according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), TAG_Stearic acid (C18:0), Normetanephrine, and Mannose provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Normetanephrine, TAG_Stearic acid (C18:0), Noradrenaline (Norepinephrine), and Behenic acid (C22:0) provided that a correction for confounders is carried out.

In another preferred embodiment of the device of the present invention said heart failure is congestive heart failure according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Mannose, Normetanephrine, Lignoceric acid (C24:0), and TAG_Stearic acid (C18:0) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Mannose, Noradrenaline (Norepinephrine), Lignoceric acid (C24:0), and TAG_Stearic acid (C18:0) provided that a correction for confounders is carried out.

In a further preferred embodiment of the device of the present invention, said heart failure is DCMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, TAG_Stearic acid (C18:0), Noradrenaline (Norepinephrine), alpha-Ketoglutarate, trans-4-Hydroxyproline, and Uric acid provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), TAG_Stearic acid (C18:0), alpha-Ketoglutarate, Uric acid, and Lignoceric acid (C24:0) provided that a correction for confounders is carried out.

In a preferred embodiment of the device of the present invention said heart failure is DCMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Uric acid, Lignoceric acid (C24:0), TAG_Stearic acid (C18:0), and Noradrenaline (Norepinephrine) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Uric acid, TAG_Stearic acid (C18:0), and Mannose provided that a correction for confounders is carried out.

In a preferred embodiment of the device of the present invention said heart failure is HCMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, TAG_Stearic acid (C18:0), Mannose, Pyruvate, and Uric acid provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, TAG_Stearic acid (C18:0), Pyruvate, Taurine, Uric acid, and Mannose provided that a correction for confounders is carried out.

In a preferred embodiment of the device of the present invention said heart failure is HCMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, Cystine, Lactate, Lignoceric acid (C24:0), alpha-Ketoglutarate, and Mannose provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Lactate, Lignoceric acid (C24:0), TAG_Stearic acid (C18:0), Cystine, and alpha-Ketoglutarate provided that a correction for confounders is carried out.

In a preferred embodiment of the device of the present invention said heart failure is HFrEF according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), TAG_Stearic acid (C18:0), Normetanephrine, and Behenic acid (C22:0) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Normetanephrine, TAG_Stearic acid (C18:0), and Behenic acid (C22:0) provided that a correction for confounders is carried out.

In a preferred embodiment of the device of the present invention said heart failure is HFrEF according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Mannose, Glycine, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, TAG_Stearic acid (C18:0), and Behenic acid (C22:0) provided that a correction for confounders is carried out.

In a preferred embodiment of the device of the present invention said heart failure is ICMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that a correction for confounders is carried out.

In a preferred embodiment of the device of the present invention said heart failure is ICMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, Behenic acid (C22:0), and Normetanephrine provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Behenic acid (C22:0), Mannose, and TAG_Stearic acid (C18:0) provided that a correction for confounders is carried out.

Furthermore, the present invention relates to a data collection comprising characteristic values of the group of biomarkers being indicative for a medical condition or effect as set forth above (i.e. diagnosing heart failure in a subject).

The term “data collection” refers to a collection of data which may be physically and/or logically grouped together. In one embodiment the physical or logical grouping is realized by classification approaches like elastic net, random forest, penalized logistic regression or others known by the person skilled in the art. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for a medical condition or effect as set forth above (e.g. a query search). Thus, if an identical or similar data set can be identified in the data collection, the test data set will be associated with the said medical condition or effect. Consequently, the information obtained from the data collection can be used, e.g., as a reference for the methods of the present invention described above. More preferably, the data collection comprises characteristic values of all biomarkers comprised by any one of the groups recited above. Also preferably, the data collection comprises scores for the group of biomarkers as set forth above.

In light of the foregoing, the present invention encompasses a data storage medium comprising the aforementioned data collection.

The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising:

(a) means for comparing characteristic values of the biomarkers of the group of biomarkers or scores of the group of biomarkers of a sample operatively linked to

(b) a data storage medium as described above.

The term “system” as used herein relates to different means which are operatively linked to each other. Said means may be implemented in a single device or may be physically separated devices which are operatively linked to each other. The means for comparing characteristic values of biomarkers, preferably, based on an algorithm for comparison as mentioned before. The data storage medium, preferably, comprises the aforementioned data collection or database, wherein each of the stored data sets being indicative for a medical condition or effect referred to above. Thus, the system of the present invention allows identifying whether a test data set is comprised by the data collection stored in the data storage medium. Consequently, the methods of the present invention can be implemented by the system of the present invention.

In a preferred embodiment of the system, means for determining characteristic values of biomarkers of a sample are comprised. The term “means for determining characteristic values of biomarkers” preferably relates to the aforementioned devices for the determination of metabolites such as mass spectrometry devices, NMR devices or devices for carrying out chemical or biological assays for the biomarkers.

Moreover, the present invention relates to a diagnostic means comprising means for the determination of at least one biomarker selected from any one of the groups referred to above.

The term “diagnostic means”, preferably, relates to a diagnostic device, system or biological or chemical assay as specified elsewhere in the description in detail.

The expression “means for the determination of at least one biomarker” refers to devices or agents which are capable of specifically recognizing the biomarker. Suitable devices may be spectrometric devices such as mass spectrometry, NMR devices or devices for carrying out chemical or biological assays for the biomarkers. Suitable agents may be compounds which specifically detect the biomarkers. Detection as used herein may be a two-step process, i.e. the compound may first bind specifically to the biomarker to be detected and subsequently generate a detectable signal, e.g., fluorescent signals, chemiluminescent signals, radioactive signals and the like. For the generation of the detectable signal further compounds may be required which are all comprised by the term “means for determination of the at least one biomarker”. Compounds which specifically bind to the biomarker are described elsewhere in the specification in detail and include, preferably, enzymes, antibodies, ligands, receptors or other biological molecules or chemicals which specifically bind to the biomarkers.

Further, the present invention relates to a diagnostic composition comprising at least the biomarkers of the group of biomarkers referred to above.

The said group of biomarkers will serve as an indicator for a medical condition or effect in the subject as set forth elsewhere herein. Thus, the biomarker molecules itself may serve as diagnostic compositions, preferably, upon visualization or detection by the means referred to in herein. Thus, a diagnostic composition which indicates the presence of a biomarker according to the present invention may also comprise the said biomarker physically, e.g., a complex of an antibody and the biomarker to be detected may serve as the diagnostic composition. Accordingly, the diagnostic composition may further comprise means for detection of the metabolites as specified elsewhere in this description. Alternatively, if detection means such as MS or NMR based techniques are used, the molecular species which serves as an indicator for the risk condition will be the at least one biomarker comprised by the test sample to be investigated. Thus, the group of biomarkers referred to in accordance with the present invention shall serve itself as a diagnostic composition due to its identification as an indicator for the disease.

In general, the present invention contemplates the use of a group of biomarkers, said group comprising Cholesterylester C18:1, Cholester-ylester C18:2, a Sphingomyelin C23:0, a Spingomyelin C24:0, and cysteine, in a sample of a subject suspected to suffer from heart failure for diagnosing heart failure or for the preparation of a pharmaceutical and/or diagnostic composition for diagnosing heart failure.

In general, the present invention contemplates the use of a group of biomarkers, said group comprising Cholesterylester C18:1, Cholester-ylester C18:2, a Sphingomyelin C23:0, a Spingomyelin C24:0, and cysteine, in a first and second sample of a subject suffering from heart failure for monitoring heart failure or for the preparation of a pharmaceutical and/or diagnostic composition for monitoring heart failure.

In a preferred embodiment of the use of the present invention, said heart failure is congestive heart failure according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), TAG_Stearic acid (C18:0), Normetanephrine, and Mannose provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Normetanephrine, TAG_Stearic acid (C18:0), Noradrenaline (Norepinephrine), and Behenic acid (C22:0) provided that a correction for confounders is carried out.

In another preferred embodiment of the use of the present invention said heart failure is congestive heart failure according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Mannose, Normetanephrine, Lignoceric acid (C24:0), and TAG_Stearic acid (C18:0) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Mannose, Noradrenaline (Norepinephrine), Lignoceric acid (C24:0), and TAG_Stearic acid (C18:0) provided that a correction for confounders is carried out.

In a further preferred embodiment of the use of the present invention, said heart failure is DCMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, TAG_Stearic acid (C18:0), Noradrenaline (Norepinephrine), alpha-Ketoglutarate, trans-4-Hydroxyproline, and Uric acid provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), TAG_Stearic acid (C18:0), alpha-Ketoglutarate, Uric acid, and Lignoceric acid (C24:0) provided that a correction for confounders is carried out.

In a preferred embodiment of the use of the present invention said heart failure is DCMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Uric acid, Lignoceric acid (C24:0), TAG_Stearic acid (C18:0), and Noradrenaline (Norepinephrine) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Uric acid, TAG_Stearic acid (C18:0), and Mannose provided that a correction for confounders is carried out.

In a preferred embodiment of the use of the present invention said heart failure is HCMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, TAG_Stearic acid (C18:0), Mannose, Pyruvate, and Uric acid provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, TAG_Stearic acid (C18:0), Pyruvate, Taurine, Uric acid, and Mannose provided that a correction for confounders is carried out.

In a preferred embodiment of the use of the present invention said heart failure is HCMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, Cystine, Lactate, Lignoceric acid (C24:0), alpha-Ketoglutarate, and Mannose provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Lactate, Lignoceric acid (C24:0), TAG_Stearic acid (C18:0), Cystine, and alpha-Ketoglutarate provided that a correction for confounders is carried out.

In a preferred embodiment of the use of the present invention said heart failure is HFrEF according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), TAG_Stearic acid (C18:0), Normetanephrine, and Behenic acid (C22:0) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Normetanephrine, TAG_Stearic acid (C18:0), and Behenic acid (C22:0) provided that a correction for confounders is carried out.

In a preferred embodiment of the use of the present invention said heart failure is HFrEF according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Mannose, Glycine, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, TAG_Stearic acid (C18:0), and Behenic acid (C22:0) provided that a correction for confounders is carried out.

In a preferred embodiment of the use of the present invention said heart failure is ICMP according to NYHA class I and
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that a correction for confounders is carried out.

In a preferred embodiment of the use of the present invention said heart failure is ICMP according to NYHA class II or III and
(i) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, Behenic acid (C22:0), and Normetanephrine provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Behenic acid (C22:0), Mannose, and TAG_Stearic acid (C18:0) provided that a correction for confounders is carried out.

The present invention also relates to a kit for carrying out the method of the present invention, said kit comprising detection agents for each of the biomarkers of the group of biomarkers comprising Cholesterylester C18:1, Cholesterylester C18:2, a Sphingomyelin C23:0, a Spingomyelin C24:0, and cysteine.

The term “kit” as used herein refers to a collection of the aforementioned components, preferably, provided separately or within a single container. The detection agents may be provided in the kit of the invention in a “ready-to-use” liquid form or in dry form. The kit may further include controls, buffers, and/or reagents. The kit also comprises instructions for carrying out the method of the present invention, as well as information on the reference values. These instructions may be in the form of a manual or may be electronically accessible information. The latter information may be provided on a data storage medium or device such as an optical storage medium (e.g., a Compact Disc) or directly on a computer or data processing device.

Suitable detection agents for the biomarkers have been specified elsewhere herein in detail. Preferably, the detection agents may be antibodies or aptameres or other molecules which are capable of binding to the biomarkers specifically.

The kit of the invention can be, preferably, used for carrying out the method of the present invention, i.e. for diagnosing heart failure as specified elsewhere herein in detail.

### FURTHER EMBODIMENTS OF THE PRESENT INVENTION

Further, is has been found in the studies underlying the present invention that the determination of a group of biomarkers as shown in Table 1C, of a group of biomarkers as shown in Table 1D, and a group of biomarkers comprising Cholesterylester C18:1, Cholesterylester C18:2, Cysteine, Isoleucine, alpha-Ketoglutarate, Sphingomyelin (d17:1,C23:0), Sphingomyelin (d17:1,C24:0), Stearic acid (C18:0) (from TAGs), and Noradrenaline allows for a reliable diagnosis of heart failure. Accordingly, the present invention envisages the following methods, uses and devices. The definitions and explanations made herein above also apply following methods, uses and devices.

The present invention further relates to a method for diagnosing heart failure in a subject comprising the steps of:
a) determining in a sample of a subject suspected to suffer from heart failure the amounts of a group of biomarkers, said group comprising: cholesterylester C18:2, sphingomyelin (d17:1,C23:0), and sphingomyelin (d17:1,C24:0); and
b) comparing the amounts of the said biomarkers to a reference, whereby heart failure is to be diagnosed.

Also, the present invention contemplates the use of a group of biomarkers, said group comprising: cholesterylester C18:2, sphingomyelin (d17:1,C23:0), and sphingomyelin (d17:1,C24:0), in a sample of a subject suspected to suffer from heart failure for diagnosing heart failure or for the preparation of a pharmaceutical and/or diagnostic composition for diagnosing heart failure.

Also, the present invention relates to a diagnostic device comprising:
a) an analysing unit comprising one at least one detector for a group of biomarkers, said group comprising: cholesterylester C18:2, sphingomyelin (d17:1,C23:0), and sphingomyelin (d17:1,C24:0), wherein said analyzing unit is adapted for determining the amounts of the said biomarkers detected by the at least one detector, and, operatively linked thereto;
b) an evaluation unit comprising a computer comprising tangibly embedded a computer program code for carrying out a comparison of the determined amounts of the group of biomarkers and reference amounts and a data base comprising said reference amounts for the said biomarkers whereby it will be diagnosed whether a subject suffers from heart failure.

Moreover, the present invention relates to a method for diagnosing heart failure in a subject comprising the steps of:
a) determining in a sample of a subject suspected to suffer from heart failure the amounts of a group of biomarkers, said group comprising: Cholesterylester C15:0, Cholesterylester C18:2, Sphingomyelin (d17:1,C23:0), and Sphingomyelin (d17:1,C24:0); and
b) comparing the amounts of the said biomarkers to a reference, whereby heart failure is to be diagnosed.

Also, the present invention contemplates the use of a group of biomarkers, said group comprising: Cholesterylester C15:0, Cholesterylester C18:2, Sphingomyelin (d17:1,C23:0), and Sphingomyelin (d17:1,C24:0), in a sample of a subject suspected to suffer from heart failure for diagnosing heart failure or for the preparation of a pharmaceutical and/or diagnostic composition for diagnosing heart failure.

Also, the present invention relates to a diagnostic device comprising:
a) an analysing unit comprising one at least one detector for a group of biomarkers, said group comprising: Cholesterylester C15:0, Cholesterylester C18:2, Sphingomyelin (d17:1,C23:0), and Sphingomyelin (d17:1,C24:0), wherein said analyzing unit is adapted for determining the amounts of the said biomarkers detected by the at least one detector, and, operatively linked thereto;
b) an evaluation unit comprising a computer comprising tangibly embedded a computer program code for carrying out a comparison of the determined amounts of the group of biomarkers and reference amounts and a data base comprising said reference amounts for the said biomarkers whereby it will be diagnosed whether a subject suffers from heart failure.

Moreover, the present invention relates to a method for diagnosing heart failure in a subject comprising the steps of:
a) determining in a sample of a subject suspected to suffer from heart failure the amounts of a group of biomarkers, said group comprising: Cholesterylester C18:1, Cholesterylester C18:2, Cysteine, Isoleucine, alpha-Ketoglutarate, Sphingomyelin (d17:1,C23:0), Sphingomyelin (d17:1,C24:0), Stearic acid (C18:0) (from TAGs), Noradrenaline; and
b) comparing the amounts of the said biomarkers to a reference, whereby heart failure is to be diagnosed.

Also, the present invention contemplates the use of a group of biomarkers, said group comprising: Cholesterylester C18:1, Cholesterylester C18:2, Cysteine, Isoleucine, alpha-Ketoglutarate, Sphingomyelin (d17:1,C23:0), Sphingomyelin (d17:1,C24:0), Stearic acid (C18:0) (from TAGs), Noradrenaline, in a sample of a subject suspected to suffer from heart failure for diagnosing heart failure or for the preparation of a pharmaceutical and/or diagnostic composition for diagnosing heart failure.

Also, the present invention relates to a diagnostic device comprising:
a) an analysing unit comprising one at least one detector for a group of biomarkers, said group comprising: Cholesterylester C18:1, Cholesterylester C18:2, Cysteine, Isoleucine, alpha-Ketoglutarate, Sphingomyelin (d17:1,C23:0), Sphingomyelin (d17:1,C24:0), Stearic acid (C18:0) (from TAGs), Noradrenaline, wherein said analyzing unit is adapted for determining the amounts of the said biomarkers detected by the at least one detector, and, operatively linked thereto;
b) an evaluation unit comprising a computer comprising tangibly embedded a computer program code for carrying out a comparison of the determined amounts of the group of biomarkers and reference amounts and a data base comprising said reference amounts for the said biomarkers whereby it will be diagnosed whether a subject suffers from heart failure.

Also it is envisaged for the above-mentioned further embodiments to determine a natriuretic peptide, such as NT-proBNP, as additional biomarker. The same applies to the use of said groups of biomarkers and to the diagnostic device.

The term heart failure has been defined herein above. The definition applies accordingly. Preferably, the heart failure to be diagnosed in connection with the further embodiments is HFrEF. More preferably, the heart failure is ICMP, in particular ICMP according to NYHA class I, II and/or III, in particular ICMP according to NYHA class II and/or III.

All references cited herein are herewith incorporated by reference with respect to their disclosure content, in general, or with respect to the specific disclosure contents indicated above.

### EXAMPLES

The invention will now be illustrated by the following Examples which are not intended to restrict or limit the scope of this invention.

### Example 1

### Study Design for the Differentiation of CHF Subtypes DCMP (Dilated Cardiomyopathy), Icmp (Ischemic Cardiomyopathy) and HCMP (Hypertrophic Cardiomyopathy) From Healthy Controls

A multicentric study with three clinical centers and in total 843 subjects was conducted. The study comprised 194 male and female DCMP-, 183 male and female ICMP- and 210 male and female HCMP patients as well as 256 male and female healthy controls in an age range from 35-75 and a BMI rage from 20-35 kg/m2. NYHA (New York Heart Association) scores of the patients ranged from 1-3. Patients and controls were matched for age, gender and BMI. For all patients and controls, a blood sample was collected. Plasma was prepared by centrifugation, and samples were stored at −80° C. until measurements were performed. Three subgroups of CHF (DCMP, ICMP and HCMP) were defined on the basis of echocardiography and hemodynamic criteria:

a) Subgroup DCMP: is hemodynamically defined as a systolic pump failure with cardiomegaly (echocardiographic enhancement of the left ventricular end diastolic diameter >55 mm and a restricted left ventricular ejection fraction—LVEF of <50%).

b) Subgroup ICMP: is hemodynamically defined as systolic pump failure due to a coronary insufficiency (>50% coronary stenosis and a stress inducible endocardium motion insufficiency as well as an LVEF of <50%)

c) Subgroup HCMP: concentric heart hypertrophy (echocardiography—septum >11 mm, posterior myocardial wall >11 mm) and with a diastolic CHF (non or mildly impaired pump function with LVEF of 50%).

NYHA IV patients were excluded as well as patients suffering from apoplex, patients who had myocardial infarction within the last 4 months before testing, patients with altered medications within the last 4 weeks before testing as well as patients who suffered from acute or chronic inflammatory diseases and malignant tumours.

### Example 2

### Determination of Metabolites

Human plasma samples were prepared and subjected to LC-MS/MS and GC-MS or SPE-LC-MS/MS (hormones) analysis as described in the following:

Proteins were separated by precipitation from blood plasma. After addition of water and a mixture of ethanol and dichlormethan the remaining sample was fractioned into an aqueous, polar phase and an organic, lipophilic phase.

For the transmethanolysis of the lipid extracts a mixture of 140 μl of chloroform, 37 μl of hydrochloric acid (37% by weight HCl in water), 320 μl of methanol and 20 μl of toluene was added to the evaporated extract. The vessel was sealed tightly and heated for 2 hours at 100° C., with shaking. The solution was subsequently evaporated to dryness. The residue was dried completely.

The methoximation of the carbonyl groups was carried out by reaction with methoxyamine hydrochloride (20 mg/ml in pyridine, 100 l for 1.5 hours at 60° C.) in a tightly sealed vessel. 20 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 100 μl of N-methyl-N-(trimethylsilyl)-2,2,2-trifluoroacetamide (MSTFA) was carried out for 30 minutes at 60° C., again in the tightly sealed vessel. The final volume before injection into the GC was 220 μl.

For the polar phase the derivatization was performed in the following way: The methoximation of the carbonyl groups was carried out by reaction with methoxyamine hydrochloride (20 mg/ml in pyridine, 50 l for 1.5 hours at 60° C.) in a tightly sealed vessel. 10 μl of a solution of odd-numbered, straight-chain fatty acids (solution of each 0.3 mg/mL of fatty acids from 7 to 25 carbon atoms and each 0.6 mg/mL of fatty acids with 27, 29 and 31 carbon atoms in 3/7 (v/v) pyridine/toluene) were added as time standards. Finally, the derivatization with 50 μl of N-methyl-N(trimethylsilyl)-2,2,2-trifluoroacetamide (MSTFA) was carried out for 30 minutes at 60° C., again in the tightly sealed vessel. The final volume before injection into the GC was 110 μl.

The GC-MS systems consisted of an Agilent 6890 GC coupled to an Agilent 5973 MSD. The autosamplers were CompiPal or GCPal from CTC.

For the analysis usual commercial capillary separation columns (30 m×0,25 mm×0,25 μm) with different poly-methyl-siloxane stationary phases containing 0% up to 35% of aromatic moieties, depending on the analysed sample materials and fractions from the phase separation step, were used (for example: DB-1 ms, HP-5ms, DB-XLB, DB-35ms, Agilent Technologies). Up to 1 μL of the final volume was injected splitless and the oven temperature program was started at 70° C. and ended at 340° C. with different heating rates depending on the sample material and fraction from the phase separation step in order to achieve a sufficient chromatographic separation and number of scans within each analyte peak. Furthermore RTL (Retention Time Locking, Agilent Technologies) was used for the analysis and usual GC-MS standard conditions, for example constant flow with nominal 1 to 1.7 ml/min. and helium as the mobile phase gas, ionisation was done by electron impact with 70 eV, scanning within a m/z range from 15 to 600 with scan rates from 2.5 to 3 scans/sec and standard tune conditions.

The HPLC-MS systems consisted of an Agilent 1100 LC system (Agilent Technologies, Waldbronn, Germany) coupled with an API 4000 Mass spectrometer (Applied Biosystem/MDS SCI-EX, Toronto, Canada). HPLC analysis was performed on commercially available reversed phase separation columns with C18 stationary phases (for example: GROM ODS 7 pH, Thermo Betasil C18). Up to 10 μL of the final sample volume of evaporated and reconstituted polar and lipophilic phase was injected and separation was performed with gradient elution using methanol/water/formic acid or acetonitrile/water/formic acid gradients at a flowrate of 200 μL/min.

Mass spectrometry was carried out by electrospray ionisation in positive mode for the non-polar fraction and negative or positive mode for the polar fraction using multiple-reaction-monitoring-(MRM)-mode and fullscan from 100-1000 amu.

Steroids and their metabolites were measured by online SPE-LC-MS (Solid phase extraction-LC-MS). Catecholamines and their metabolites were measured by online SPE-LC-MS as described by Yamada et al. (J. Anal. Toxicol. (26), 2002, 17-22). For both catecholamines and related metabolites and steroids and related metabolites, quantification was achieved by means of stable-isotope-labelled standards, and absolute concentrations were calculated.

Analysis of complex lipids in plasma samples:

Total lipids were extracted from plasma by liquid/liquid extraction using chloroform/methanol. The lipid extracts were subsequently fractionated by normal phase liquid chromatography (NPLC) into eleven different lipid groups according to Christie (Journal of Lipid Research (26), 1985, 507-512).

The fractions were analyzed by LC-MS/MS using electrospray ionization (ESI) and atmospheric pressure chemical ionization (APCI) with detection of specific multiple reaction monitoring (MRM) transitions for cholesterol esters (CE), free sterols (FS), sphingoymelins (SM), and ceramides (CER) respectively. Sphingosines and sphingosine-1-phosphates (SP) were analyzed by LC-MS/MS using electrospray ionization (ESI) with detection of specific multiple reaction monitoring (MRM) transitions as described by Schmidt H et al., Prostaglandins & other Lipid Mediators 81(2006), 162-170. Metabolites in the Tables below are derived from one of these fractions include the respective abbreviation in their name.

The lipid classes Monoacylglycerides (MAG), Triacylglycerides (TAG), Phosphatidylcholines (PC), Phosphatidylserines (PS), Phosphatidylinositoles (PI), Lysophosphatidylcholines (LPC), Diacylglycerols (DAG), Free fatty acids (FFA) were measured by GC-MS.

The fractions were analyzed by GC-MS after derivatization with TMSH (Trimethyl sulfonium hydroxide), yielding the fatty acid methyl esters (FAME) corresponding to the acyl moieties of the class-separated lipids. The concentrations of FAME from C14 to C24 were determined in each fraction.

Metabolites in the Tables below derived from one of these fractions include the respective abbreviation in front of their name separated by an underscore. For example, TAG_Stearic acid (C18:0) means TAG (Triacylglycerides) wherein at least a fatty acid moiety of the triacylglyceride is Stearic acid (C:18:0).

Eicosanoids and related were measured out of plasma by offline- and online-SPE LC-MS/MS (Solid phase extraction-LC-MS/MS) (Masoodi M and Nicolaou A: Rapid Commun Mass Spectrom. 2006; 20(20): 3023-3029. Absolute quantification was performed by means of stable isotope-labelled standards.

Samples from all patients were subjected to the full method spectrum of metabolite profiling analyses as described above, with the exception of metabolite profiling of the polar phase of plasma by LC-MS/MS using positive electrospray ionisation mode which was applied to a subset of 75 samples comprising controls, DCMP NYHA I, DCMP NYHA III, HCMP NYHA II and ICMP NYHA III patients.

### Example 3

### Data Analysis and Statistical Evaluation

Plasma samples were analyzed in randomized analytical sequence design with pooled samples (so called “pool”) generated from aliquots of each sample. Following comprehensive analytical validation steps, the raw peak data for each analyte were normalized to the median of pool per analytical sequence to account for process variability (so called “pool-normalized ratios”). If available, absolute concentrations of metabolites were used for statistical analysis. In all other cases, pool-normalized ratios were used. All data were log10-transformed to achieve normal distribution.

The data of the study described in Example 1 were utilized for the identification of multimarker panels for the classification of CHF subgroups compared to healthy controls. Metabolite data were corrected for confounding factors or uncorrected metabolite data were used. The ANOVA model for correction for confounders comprised the factors age, BMI, gender and CHF subgroup (ANOVA model: CHF_SUBGROUP+(GENDER+AGE+BMI)̂2; correction factors: GENDER, AGE and BMI). CHF patients were subdivided based on a combination of NYHA class (I or II-III) and CHF subtype (DCMP, HCMP, ICMP or the joined DCMP+ICMP group named HfrEF (heart failure with reduced ejection fraction)).

DCMP and ICMP defines heart failure with a systolic dysfunction and reduced ejection fraction of the left ventricle and therefore the combined group called heart failure with reduced ejection fraction (HfrEF) was included in the analysis as well. In contrast, HCMP defines heart failure with a diastolic dysfunction with preserved ejection fraction of the left ventricle and therefore this group can be called heart failure with preserved ejection fraction (HfpEF) as well.

For these subgroups, multi-marker panels were defined. These panels comprised at least 5 metabolites, referred to as core panel (see Table 1A for basic composition of core panels and Table 1B for a metabolite selection for core panel composition). This (minimal) core panel, as shown in Table 1A, comprises cholesterylester C18:1, cholesterylester C18:2, cysteine and each one sphingomyelin with a C23 fatty acid and one with a C24 fatty acid, respectively. With regard to this basic core panel composition, a similar performance was observed for all possible permutations of sphingomyelins with C23 or C24 fatty acid constituents (see Table 3). A specific, further reduced subset of the core panel as described above is shown in Table 10 and comprises cholesterylester C18:2, sphingomyelin (d17:1,C23:0), and sphingomyelin (d17:1,C24:0).

Another panel is shown in Table 1D and comprises Cholesterylester C15:0, Cholesterylester C18:2, Sphingomyelin (d17:1,C23:0), and Sphingomyelin (d17:1,C24:0).

For the performance calculations only two representative sphingomyelins for each fatty acid constituent were included in the core panel as shown in Table 1A, so the 5 metabolites of the core panel were identical for each classification task for all CHF subgroups (see Table 4B).

The metabolites “isoleucine” and “Sphingomyelin (d18:1, C21:0)” from Table 2 have been shown to be of value for use in combination with the known marker NT-proBNP (data not shown).

Extended panels of 10 metabolites as shown in Table 4A were created from a core panel as described above by addition of metabolites selected from the ones contained in Table 2. The additional metabolites of the extended list (Table 2) were selected with an elastic net specifically for each CHF subgroup and classification task; for an overview of the metabolites for each subgroup and classification task see Table 4A.

Furthermore, the following extended panel of 9 metabolites showed good performance in combination with the known marker NT-proBNP, in particular for ICMP (NYHA I) or DCMP (NYHA I) (data not shown):

Cholesterylester C18:1, Cholesterylester C18:2, Cysteine, Isoleucine, alpha-Ketoglutarate, Sphingomyelin (d17:1,C23:0), Sphingomyelin (d17:1,C24:0), Stearic acid (C18:0) (from TAGs), Noradrenaline

A classifier was built with an elastic net with these sets of metabolites and the cross validated classification performance was estimated with the area under the curve (AUC) of a receiver operating characteristic (ROC) analysis. Performance calculations were carried out with or without prior ANOVA correction of metabolite data for confounding factors (age, gender, BMI).

The performance of the multi-marker panels was assessed by comparison to multimarker panels consisting of the best metabolites (based on ANOVA p-value; Tables 5A and 5B, respectively) or to randomly chosen metabolites that had also been regarded/validated in the study as described in example 1 herein above, for each subgroup from WO2013/014286.

In this context and in the following, “best” metabolites from WO2013/014286 is meant to refer to the (5 or 10) best metabolites that have also been assessed in the course of the study described above; i. e., some of the metabolites from WO2013/014286 have been excluded to ensure comparability of the respective datasets used for classifier calculation.

More precisely, the extended panels of 10 metabolites as shown in Table 4A were compared to panels of the best 10 metabolites as found in WO2013/014286 and shown in Table 5A and to a respective random selection (panels not shown), and the core panel of 5 metabolites as shown in Table 4B was compared to panels of the best 5 metabolites as found in WO2013/014286 and shown in Table 5B and to a respective random selection (panels not shown).

The best metabolites or the randomly chosen metabolites were taken from the CHF subgroup specific lists of significantly deregulated metabolites (up- or downregulated; ANOVA p-value <0.05), respectively, and the same approach for classification (elastic net) was applied as for the multimarker panels of the invention. For the panels with randomly chosen metabolites the mean performance of 10 cross-validated variants of randomly chosen metabolites for each classification task was calculated. Performance calculation for DCMP NYHA I was not possible for the best metabolites or the randomly chosen metabolites due to a too low number of significantly deregulated metabolites in WO2013/014286 for this subgroup.

In general, a superior performance was observed with multimarker panels according to the invention as compared to the best metabolites or the randomly chosen metabolites from WO2013/014286. For an overview of the performance for the classifiers for each subgroup and classification task see Table 6 (core panel) and Table 7 (core panel plus metabolites from the extended panel).

For the panels as shown in Tables 1C and 1D, respectively, high performance values were obtained even without the addition of further metabolites—especially when combined with the known marker NT-proBNP. This was especially the case with regard to the CHF-subgroup HFrEF, and here especially ICMP (NYHA classes I, II, and III).

In the following tables, AUC (area under the curve) values indicate classification performance estimated by cross validated ROC (receiver operating characteristic) analysis on the data set described in Example 1. The higher the AUC value, the better is the classification performance.

TABLE 1A
Basic core panel composition.

| Metabolite | Regulation |
|---|---|
| Cholesterylester C18:1 | down |
| Cholesterylester C18:2 | down |
| Sphingomyelin (C23:0) | down |
| Sphingomyelin (C24:0) | down |
| Cysteine | up |

TABLE 1B
Metabolites for core panel composition.

| Metabolite | Regulation |
|---|---|
| Cholesterylester C18:1 | down |
| Cholesterylester C18:2 | down |
| Sphingomyelin (d16:1, C23:0) | down |
| Sphingomyelin (d16:1, C24:0) | down |
| Sphingomyelin (d17:1, C24:0) | down |
| Sphingomyelin (d17:1, C23:0) | down |
| Sphingomyelin (d18:1, C23:0) | down |
| Sphingomyelin (d18:1, C24:0) | down |
| Sphingomyelin (d18:2, C23:0) | down |
| Sphingomyelin (d18:2, C24:0) | down |
| Cysteine | up |

TABLE 1C
Further panel variant.

| Metabolite | Regulation |
|---|---|
| Cholesterylester C18:2 | down |
| Sphingomyelin (d17:1, C23:0) | down |
| Sphingomyelin (d17:1, C24:0) | down |

TABLE 1D
Further panel variant.

| Metabolite | Regulation |
|---|---|
| Cholesterylester C15:0 | down |
| Cholesterylester C18:2 | down |
| Sphingomyelin (d17:1, C23:0) | down |
| Sphingomyelin (d17:1, C24:0) | down |

TABLE 2
Extended list of metabolites for classification (with
or without ANOVA correction for confounders).

| | Without | With | |
|---|---|---|---|
| | ANOVA for | ANOVA for | |
| Metabolite | confounders | confounders | Regulation |
| 4-Hydroxy-3-methoxyphenyl- | X | X | up |
| glycol (HMPG) | | | |
| alpha-Ketoglutarate | X | X | up |
| Behenic acid (C22:0) | X | X | down |
| Cystine | X | X | up |
| Glycine | X | | down |
| Isoleucine | | | up |
| Lactate | X | X | up |
| Lignoceric acid (C24:0) | X | X | down |
| Mannose | X | X | up |
| Noradrenaline (Norepinephrine) | X | X | up |
| Normetanephrine | X | X | up |
| Pyruvate | X | X | up |
| Sphingomyelin (d18:1, C21:0) | | | down |
| TAG_Stearic acid (C18:0) | X | X | up |
| Taurine | | X | up |
| trans-4-Hydroxyproline | X | X | up |
| Uric acid | X | X | up |

TABLE 3
Performance of different core panel combinations (5 metabolites)
regarding C23 and C24 constituents of sphingomyelins for all
CHF samples (CHF group: all samples) compared with controls
(with or without ANOVA correction for confounders).

| | ANOVA cor- | | Sphingomyelin | Sphingomyelin |
|---|---|---|---|---|
| | rection for | | with C23 | with C24 |
| Panel | confounders | AUC | fatty acid | fatty acid |
| 1 | YES | 0.7904862 | Sphingomyelin | Sphingomyelin |
| | | | (d16:1, C23:0) | (d16:1, C24:0) |
| 2 | YES | 0.79595296 | Sphingomyelin | Sphingomyelin |
| | | | (d16:1, C23:0) | (d17:1, C24:0) |
| 3 | YES | 0.79672484 | Sphingomyelin | Sphingomyelin |
| | | | (d16:1, C23:0) | (d18:1, C24:0) |
| 4 | YES | 0.79043758 | Sphingomyelin | Sphingomyelin |
| | | | (d16:1, C23:0) | (d18:2, C24:0) |
| 5 | YES | 0.78910775 | Sphingomyelin | Sphingomyelin |
| | | | (d17:1, C23:0) | (d16:1, C24:0) |
| 6 | YES | 0.7955126 | Sphingomyelin | Sphingomyelin |
| | | | (d17:1, C23:0) | (d17:1, C24:0) |
| 7 | YES | 0.79481605 | Sphingomyelin | Sphingomyelin |
| | | | (d17:1, C23:0) | (d18:1, C24:0) |
| 8 | YES | 0.79035781 | Sphingomyelin | Sphingomyelin |
| | | | (d17:1, C23:0) | (d18:2, C24:0) |
| 9 | YES | 0.79289743 | Sphingomyelin | Sphingomyelin |
| | | | (d18:1, C23:0) | (d16:1, C24:0) |
| 10 | YES | 0.7956618 | Sphingomyelin | Sphingomyelin |
| | | | (d18:1, C23:0) | (d17:1, C24:0) |
| 11 | YES | 0.78288646 | Sphingomyelin | Sphingomyelin |
| | | | (d18:1, C23:0) | (d18:1, C24:0) |
| 12 | YES | 0.78358425 | Sphingomyelin | Sphingomyelin |
| | | | (d18:1, C23:0) | (d18:2, C24:0) |
| 13 | YES | 0.78870162 | Sphingomyelin | Sphingomyelin |
| | | | (d18:2, C23:0) | (d16:1, C24:0) |
| 14 | YES | 0.79550384 | Sphingomyelin | Sphingomyelin |
| | | | (d18:2, C23:0) | (d17:1, C24:0) |
| 15 | YES | 0.78539292 | Sphingomyelin | Sphingomyelin |
| | | | (d18:2, C23:0) | (d18:1, C24:0) |
| 16 | YES | 0.77647553 | Sphingomyelin | Sphingomyelin |
| | | | (d18:2, C23:0) | (d18:2, C24:0) |
| 1 | NO | 0.83722322 | Sphingomyelin | Sphingomyelin |
| | | | (d16:1, C23:0) | (d16:1, C24:0) |
| 2 | NO | 0.84191245 | Sphingomyelin | Sphingomyelin |
| | | | (d16:1, C23:0) | (d17:1, C24:0) |
| 3 | NO | 0.84296466 | Sphingomyelin | Sphingomyelin |
| | | | (d16:1, C23:0) | (d18:1, C24:0) |
| 4 | NO | 0.83773911 | Sphingomyelin | Sphingomyelin |
| | | | (d16:1, C23:0) | (d18:2, C24:0) |
| 5 | NO | 0.83704957 | Sphingomyelin | Sphingomyelin |
| | | | (d17:1, C23:0) | (d16:1, C24:0) |
| 6 | NO | 0.84189961 | Sphingomyelin | Sphingomyelin |
| | | | (d17:1, C23:0) | (d17:1, C24:0) |
| 7 | NO | 0.84317813 | Sphingomyelin | Sphingomyelin |
| | | | (d17:1, C23:0) | (d18:1, C24:0) |
| 8 | NO | 0.83867802 | Sphingomyelin | Sphingomyelin |
| | | | (d17:1, C23:0) | (d18:2, C24:0) |
| 9 | NO | 0.83955173 | Sphingomyelin | Sphingomyelin |
| | | | (d18:1, C23:0) | (d16:1, C24:0) |
| 10 | NO | 0.84180518 | Sphingomyelin | Sphingomyelin |
| | | | (d18:1, C23:0) | (d17:1, C24:0) |
| 11 | NO | 0.83860689 | Sphingomyelin | Sphingomyelin |
| | | | (d18:1, C23:0) | (d18:1, C24:0) |
| 12 | NO | 0.8366693 | Sphingomyelin | Sphingomyelin |
| | | | (d18:1, C23:0) | (d18:2, C24:0) |
| 13 | NO | 0.83699581 | Sphingomyelin | Sphingomyelin |
| | | | (d18:2, C23:0) | (d16:1, C24:0) |
| 14 | NO | 0.84078414 | Sphingomyelin | Sphingomyelin |
| | | | (d18:2, C23:0) | (d17:1, C24:0) |
| 15 | NO | 0.83937918 | Sphingomyelin | Sphingomyelin |
| | | | (d18:2, C23:0) | (d18:1, C24:0) |
| 16 | NO | 0.83363363 | Sphingomyelin | Sphingomyelin |
| | | | (d18:2, C23:0) | (d18:2, C24:0) |

TABLE 4A
Extended panels (10 metabolites), as used for performance/classifier
calculation for each CHF subgroup (with or without
ANOVA correction for confounders).

| | ANOVA | |
|---|---|---|
| | Cor- | |
| Group | rection | Metabolite |
| CHF | NO | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | alpha-Ketoglutarate |
| | | Noradrenaline (Norepinephrine) |
| | | TAG_Stearic acid (C18:0) |
| | | Normetanephrine |
| | | Mannose |
| CHF | NO | Sphingomyelin (d17:1, C24:0) |
| (NYHA II or III) | | Cholesterylester C18:2 |
| | | Sphingomyelin (d17:1, C23:0) |
| | | Cysteine |
| | | Cholesterylester C18:1 |
| | | alpha-Ketoglutarate |
| | | Mannose |
| | | Normetanephrine |
| | | Lignoceric acid (C24:0) |
| | | TAG_Stearic acid (C18:0) |
| DCMP | NO | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | TAG_Stearic acid (C18:0) |
| | | Noradrenaline (Norepinephrine) |
| | | alpha-Ketoglutarate |
| | | trans-4-Hydroxyproline |
| | | Uric acid |
| DCMP | NO | Sphingomyelin (d17:1, C24:0) |
| (NYHA II or III) | | Cholesterylester C18:2 |
| | | Sphingomyelin (d17:1, C23:0) |
| | | Cysteine |
| | | Cholesterylester C18:1 |
| | | alpha-Ketoglutarate |
| | | Uric acid |
| | | Lignoceric acid (C24:0) |
| | | TAG_Stearic acid (C18:0) |
| | | Noradrenaline (Norepinephrine) |
| HCMP | NO | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | alpha-Ketoglutarate |
| | | TAG_Stearic acid (C18:0) |
| | | Mannose |
| | | Pyruvate |
| | | Uric acid |
| HCMP | NO | Sphingomyelin (d17:1, C24:0) |
| (NYHA II or III) | | Cholesterylester C18:2 |
| | | Sphingomyelin (d17:1, C23:0) |
| | | Cysteine |
| | | Cholesterylester C18:1 |
| | | Cystine |
| | | Lactate |
| | | Lignoceric acid (C24:0) |
| | | alpha-Ketoglutarate |
| | | Mannose |
| HFrEF | NO | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | alpha-Ketoglutarate |
| | | Noradrenaline (Norepinephrine) |
| | | TAG_Stearic acid (C18:0) |
| | | Normetanephrine |
| | | Behenic acid (C22:0) |
| HFrEF | NO | Sphingomyelin (d17:1, C24:0) |
| (NYHA II or III) | | Cholesterylester C18:2 |
| | | Sphingomyelin (d17:1, C23:0) |
| | | Cysteine |
| | | Cholesterylester C18:1 |
| | | alpha-Ketoglutarate |
| | | Noradrenaline (Norepinephrine) |
| | | Mannose |
| | | Glycine |
| | | trans-4-Hydroxyproline |
| ICMP | NO | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | alpha-Ketoglutarate |
| | | 4-Hydroxy-3-methoxyphenylglycol |
| | | (HMPG) |
| | | Behenic acid (C22:0) |
| | | Lactate |
| | | trans-4-Hydroxyproline |
| ICMP NYHA | NO | Sphingomyelin (d17:1, C24:0) |
| (II or III) | | Cholesterylester C18:2 |
| | | Sphingomyelin (d17:1, C23:0) |
| | | Cysteine |
| | | Cholesterylester C18:1 |
| | | Noradrenaline (Norepinephrine) |
| | | alpha-Ketoglutarate |
| | | Mannose |
| | | Behenic acid (C22:0) |
| | | Normetanephrine |
| CHF | YES | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | alpha-Ketoglutarate |
| | | Normetanephrine |
| | | TAG_Stearic acid (C18:0) |
| | | Noradrenaline (Norepinephrine) |
| | | Behenic acid (C22:0) |
| CHF | YES | Sphingomyelin (d17:1, C24:0) |
| (NYHA II or III) | | Cholesterylester C18:2 |
| | | Sphingomyelin (d17:1, C23:0) |
| | | Cysteine |
| | | Cholesterylester C18:1 |
| | | alpha-Ketoglutarate |
| | | Mannose |
| | | Noradrenaline (Norepinephrine) |
| | | Lignoceric acid (C24:0) |
| | | TAG_Stearic acid (C18:0) |
| DCMP | YES | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | Noradrenaline (Norepinephrine) |
| | | TAG_Stearic acid (C18:0) |
| | | alpha-Ketoglutarate |
| | | Uric acid |
| | | Lignoceric acid (C24:0) |
| DCMP | YES | Sphingomyelin (d17:1, C24:0) |
| (NYHA II or III) | | Cholesterylester C18:2 |
| | | Sphingomyelin (d17:1, C23:0) |
| | | Cysteine |
| | | Cholesterylester C18:1 |
| | | alpha-Ketoglutarate |
| | | Noradrenaline (Norepinephrine) |
| | | Uric acid |
| | | TAG_Stearic acid (C18:0) |
| | | Mannose |
| HCMP | YES | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | TAG_Stearic acid (C18:0) |
| | | Pyruvate |
| | | Taurine |
| | | Uric acid |
| | | Mannose |
| HCMP | YES | Sphingomyelin (d17:1, C24:0) |
| (NYHA II or III) | | Cholesterylester C18:2 |
| | | Sphingomyelin (d17:1, C23:0) |
| | | Cysteine |
| | | Cholesterylester C18:1 |
| | | Lactate |
| | | Lignoceric acid (C24:0) |
| | | TAG_Stearic acid (C18:0) |
| | | Cystine |
| | | alpha-Ketoglutarate |
| HFrEF | YES | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | alpha-Ketoglutarate |
| | | Noradrenaline (Norepinephrine) |
| | | Normetanephrine |
| | | TAG_Stearic acid (C18:0) |
| | | Behenic acid (C22:0) |
| HFrEF | YES | Sphingomyelin (d17:1, C24:0) |
| (NYHA II or III) | | Cholesterylester C18:2 |
| | | Sphingomyelin (d17:1, C23:0) |
| | | Cysteine |
| | | Cholesterylester C18:1 |
| | | Noradrenaline (Norepinephrine) |
| | | alpha-Ketoglutarate |
| | | Mannose |
| | | TAG_Stearic acid (C18:0) |
| | | Behenic acid (C22:0) |
| ICMP | YES | Sphingomyelin (d17:1, C24:0) |
| (NYHA I) | | Cholesterylester C18:2 |
| | | Sphingomyelin (d17:1, C23:0) |
| | | Cysteine |
| | | Cholesterylester C18:1 |
| | | alpha-Ketoglutarate |
| | | 4-Hydroxy-3-methoxyphenylglycol |
| | | (HMPG) |
| | | Behenic acid (C22:0) |
| | | Lactate |
| | | trans-4-Hydroxyproline |
| ICMP | YES | Sphingomyelin (d17:1, C24:0) |
| (NYHA II or III) | | Cholesterylester C18:2 |
| | | Sphingomyelin (d17:1, C23:0) |
| | | Cysteine |
| | | Cholesterylester C18:1 |
| | | Noradrenaline (Norepinephrine) |
| | | alpha-Ketoglutarate |
| | | Behenic acid (C22:0) |
| | | Mannose |
| | | TAG_Stearic acid (C18:0) |

TABLE 4B
Core panel of 5 metabolites, as used for performance/classifier
calculation for each CHF subgroup, both with and without
ANOVA correction for confounders.

| Group | Metabolites |
|---|---|
| All groups | Sphingomyelin (d17:1, C24:0) |
| | Cholesterylester C18:2 |
| | Sphingomyelin (d17:1, C23:0) |
| | Cysteine |
| | Cholesterylester C18:1 |

TABLE 5A
Metabolite panels (n = 10) as selected from the corresponding
best metabolites (based on p-value of ANOVA) of WO2013/014286 for
performance/classifier calculation, both with and without ANOVA.

| Group | Metabolites |
|---|---|
| CHF | Cholesterylester C18:2 |
| (NYHA I) | Cholesterylester C15:0 |
| | Sphingomyelin (d18:1, C23:0) |
| | Sphingomyelin (d17:1, C23:0) |
| | Tricosanoic acid (C23:0) |
| | Taurine |
| | Cholesterylester C18:1 |
| | TAG (C18:1, C18:2) |
| | Sphingomyelin (d18:1, C14:0) |
| | Sphingomyelin (d18:2, C23:0) |
| CHF | Sphingomyelin (d17:1, C24:0) |
| (NYHA II or III) | Tricosanoic acid (C23:0) |
| | Cholesterylester C18:2 |
| | Cholesterylester C15:0 |
| | Sphingomyelin (d16:1, C23:0) |
| | Sphingomyelin (d17:1, C23:0) |
| | Sphingomyelin (d17:1, C22:0) |
| | Sphingomyelin (d18:1, C23:0) |
| | Sphingomyelin (d18:2, C23:0) |
| | 1-Hydroxy-2-amino-(cis, trans)-3,5- |
| | octadecadiene (from sphingolipids) |
| DCMP | threo-Sphingosine (d18:1) |
| (NYHA I) | erythro-Dihydrosphingosine (d16:0) |
| | Proline |
| | 5-O-Methylsphingosine (d18:1) |
| | TAG (C16:0, C18:1, C18:2) |
| DCMP | Sphingomyelin (d17:1, C24:0) |
| (NYHA II or III) | Sphingomyelin (d16:1, C23:0) |
| | Sphingomyelin (d17:1, C22:0) |
| | Sphingomyelin (d17:1, C23:0) |
| | Tricosanoic acid (C23:0) |
| | 1-Hydroxy-2-amino-(cis, trans)-3,5- |
| | octadecadiene (from sphingolipids) |
| | Sphingomyelin (d16:1, C22:0) |
| | Isocitrate |
| | Sphingomyelin (d17:1, C20:0) |
| | erythro-Dihydrosphingosine (d16:0) |
| HCMP | Sphingosine (d16:1) |
| (NYHA I) | Sphingadienine (d18:2) |
| | TAG (C18:1, C18:2) |
| | Cholesterylester C20:4 |
| | TAG (C16:0, C18:1, C18:2) |
| | Pyruvate |
| | Sphingadienine-1-phosphate (d18:2) |
| | TAG (C16:0, C16:1) |
| | Ketoleucine |
| | TAG (C18:2, C18:2) |
| HCMP | Cholesterylester C18:2 |
| (NYHA II or III) | Cysteine |
| | Sphingomyelin (d18:0, C18:0) |
| | Uric acid |
| | Pyruvate |
| | Taurine |
| | Cholesterylester C18:1 |
| | Sphingosine (d16:1) |
| | Ceramide (d18:1, C18:0) |
| | Cystine |
| HFrEF | TAG (C18:1, C18:2) |
| (NYHA I) | TAG (C16:0, C18:1, C18:2) |
| | threo-Sphingosine (d18:1) |
| | Cholesterylester C20:1 |
| | Sphingomyelin (d18:2, C23:1) |
| | Sphingosine-1-phosphate (d17:1) |
| | Sphingadienine-1-phosphate (d18:2) |
| | Sphingomyelin (d18:2, C24:2) |
| | Sphingomyelin (d18:1, C16:0) |
| | Sphingomyelin (d18:1, C23:1) |
| HFrEF | Sphingomyelin (d17:1, C24:0) |
| (NYHA II or III) | Sphingomyelin (d16:1, C23:0) |
| | Sphingomyelin (d17:1, C22:0) |
| | Sphingomyelin (d17:1, C23:0) |
| | Cholesterylester C18:2 |
| | Tricosanoic acid (C23:0) |
| | 1-Hydroxy-2-amino-(cis, trans)-3,5- |
| | octadecadiene (from sphingolipids) |
| | Sphingomyelin (d16:1, C22:0) |
| | Isocitrate |
| | Sphingomyelin (d17:1, C20:0) |
| ICMP | TAG (C18:1, C18:2) |
| (NYHA I) | TAG (C16:0, C18:1, C18:2) |
| | threo-Sphingosine (d18:1) |
| | Cholesterylester C20:1 |
| | Sphingomyelin (d18:2, C23:1) |
| | Sphingosine-1-phosphate (d17:1) |
| | Sphingadienine-1-phosphate (d18:2) |
| | Sphingomyelin (d18:2, C24:2) |
| | Sphingomyelin (d18:1, C16:0) |
| | Sphingomyelin (d18:1, C23:1) |
| ICMP | Cholesterylester C18:2 |
| (NYHA II or III) | Sphingomyelin (d17:1, C24:0) |
| | Sphingomyelin (d18:2, C23:0) |
| | Sphingomyelin (d17:1, C23:0) |
| | Tricosanoic acid (C23:0) |
| | Sphingomyelin (d16:1, C23:0) |
| | Sphingomyelin (d18:1, C14:0) |
| | 1-Hydroxy-2-amino-(cis, trans)-3,5- |
| | octadecadiene (from sphingolipids) |
| | Cholesterylester C15:0 |
| | Sphingomyelin (d17:1, C24:1) |

TABLE 5B
Metabolite panels (5 metabolites) as selected from
the corresponding best metabolites (based on p-value
of ANOVA) of WO2013/014286 for performance/classifier
calculation, both with and without ANOVA.

| Group | Metabolites |
|---|---|
| CHF | Cholesterylester C18:2 |
| (NYHA I) | Cholesterylester C15:0 |
| | Sphingomyelin (d18:1, C23:0) |
| | Sphingomyelin (d17:1, C23:0) |
| | Tricosanoic acid (C23:0) |
| CHF | Sphingomyelin (d17:1, C24:0) |
| (NYHA II or III) | Tricosanoic acid (C23:0) |
| | Cholesterylester C18:2 |
| | Cholesterylester C15:0 |
| | Sphingomyelin (d16:1, C23:0) |
| DCMP | threo-Sphingosine (d18:1) |
| (NYHA I) | erythro-Dihydrosphingosine (d16:0) |
| | Proline |
| | 5-O-Methylsphingosine (d18:1) |
| | TAG (C16:0, C18:1, C18:2) |
| DCMP | Sphingomyelin (d17:1, C24:0) |
| (NYHA II or III) | Sphingomyelin (d16:1, C23:0) |
| | Sphingomyelin (d17:1, C22:0) |
| | Sphingomyelin (d17:1, C23:0) |
| | Tricosanoic acid (C23:0) |
| HCMP | Sphingosine (d16:1) |
| (NYHA I) | Sphingadienine (d18:2) |
| | TAG (C18:1, C18:2) |
| | Cholesterylester C20:4 |
| | TAG (C16:0, C18:1, C18:2) |
| HCMP | Cholesterylester C18:2 |
| (NYHA II or III) | Cysteine |
| | Sphingomyelin (d18:0, C18:0) |
| | Uric acid |
| | Pyruvate |
| HFrEF | TAG (C18:1, C18:2) |
| (NYHA I) | TAG (C16:0, C18:1, C18:2) |
| | threo-Sphingosine (d18:1) |
| | Cholesterylester C20:1 |
| | Sphingomyelin (d18:2, C23:1) |
| HFrEF | Sphingomyelin (d17:1, C24:0) |
| (NYHA II or III) | Sphingomyelin (d16:1, C23:0) |
| | Sphingomyelin (d17:1, C22:0) |
| | Sphingomyelin (d17:1, C23:0) |
| | Cholesterylester C18:2 |
| ICMP | TAG (C18:1, C18:2) |
| (NYHA I) | TAG (C16:0, C18:1, C18:2) |
| | threo-Sphingosine (d18:1) |
| | Cholesterylester C20:1 |
| | Sphingomyelin (d18:2, C23:1) |
| ICMP | Cholesterylester C18:2 |
| (NYHA II or III) | Sphingomyelin (d17:1, C24:0) |
| | Sphingomyelin (d18:2, C23:0) |
| | Sphingomyelin (d17:1, C23:0) |
| | Tricosanoic acid (C23:0) |

TABLE 6
Performance comparison of a core panel as shown in Table
4B to the corresponding five best metabolites of WO2013/014286
as shown in Table 5B and five randomly chosen metabolites
of WO2013/014286, respectively (with or without ANOVA
correction for confounders).

| | | AUC: | AUC: | AUC: |
|---|---|---|---|---|
| | | Core | Best 5 | 5 random |
| ANOVA | | Panel ac- | Metabolites | Metabolites |
| Cor- | CHF | cording to | from WO2013/ | from WO2013/ |
| rection | Subgroup | Invention | 014286 | 014286 |
| NO | CHF | 0.80789 | 0.75891 | 0.71267 |
| | (NYHA I) | | | |
| NO | CHF (NYHA | 0.87517 | 0.83738 | 0.79201 |
| | II or III) | | | |
| NO | DCMP | 0.76919 | 0.71441 | |
| | (NYHA I) | | | |
| NO | DCMP (NYHA | 0.86349 | 0.78299 | 0.7508 |
| | II or III) | | | |
| NO | HCMP | 0.76894 | 0.52396 | 0.67287 |
| | (NYHA I) | | | |
| NO | HCMP (NYHA | 0.77252 | 0.79076 | 0.72577 |
| | II or III) | | | |
| NO | HFrEF | 0.83292 | 0.80533 | 0.78485 |
| | (NYHA I) | | | |
| NO | HFrEF (NYHA | 0.90334 | 0.87548 | 0.79981 |
| | II or III) | | | |
| NO | ICMP | 0.88928 | 0.87598 | 0.83807 |
| | (NYHA I) | | | |
| NO | ICMP (NYHA | 0.9367 | 0.91023 | 0.84099 |
| | II or III) | | | |
| YES | CHF | 0.74363 | 0.71458 | 0.66638 |
| | (NYHA I) | | | |
| YES | CHF (NYHA | 0.835 | 0.81676 | 0.77156 |
| | II or III) | | | |
| YES | DCMP | 0.72963 | 0.61612 | |
| | (NYHA I) | | | |
| YES | DCMP (NYHA | 0.82744 | 0.77077 | 0.71285 |
| | II or III) | | | |
| YES | HCMP | 0.65328 | 0.51885 | 0.6125 |
| | (NYHA I) | | | |
| YES | HCMP (NYHA | 0.72186 | 0.66671 | 0.67054 |
| | II or III) | | | |
| YES | HFrEF | 0.7799 | 0.76601 | 0.73797 |
| | (NYHA I) | | | |
| YES | HFrEF (NYHA | 0.86687 | 0.84505 | 0.74078 |
| | II or III) | | | |
| YES | ICMP | 0.83474 | 0.85344 | 0.79594 |
| | (NYHA I) | | | |
| YES | ICMP (NYHA | 0.89707 | 0.87674 | 0.78758 |
| | II or III) | | | |

TABLE 7
Performance comparison of extended panels as shown in
Table 4A to the corresponding ten best metabolites from
WO13014286 as shown in Table 5A and ten randomly chosen
metabolites of WO2013/014286, respectively (with or
without ANOVA correction for confounders).

| | | AUC: | AUC: | AUC: |
|---|---|---|---|---|
| | | Extended | Best 10 | 10 random |
| ANOVA | | Panel ac- | Metabolites | Metabolites |
| Cor- | CHF | cording to | from WO2013/ | from WO2013/ |
| rection | Subgroup | Invention | 014286 | 014286 |
| NO | CHF | 0.83952 | 0.77511 | 0.75408 |
| | (NYHA I) | | | |
| NO | CHF (NYHA | 0.91262 | 0.8358 | 0.81833 |
| | II or III) | | | |
| NO | DCMP | 0.83933 | | |
| | (NYHA I) | | | |
| NO | DCMP (NYHA | 0.86968 | 0.78276 | 0.81317 |
| | II or III) | | | |
| NO | HCMP | 0.78575 | 0.67637 | 0.69865 |
| | (NYHA I) | | | |
| NO | HCMP (NYHA | 0.88339 | 0.84966 | 0.74474 |
| | II or III) | | | |
| NO | HFrEF | 0.87768 | 0.81451 | 0.81411 |
| | (NYHA I) | | | |
| NO | HFrEF (NYHA | 0.92836 | 0.88711 | 0.83503 |
| | II or III) | | | |
| NO | ICMP | 0.92844 | 0.88305 | 0.87486 |
| | (NYHA I) | | | |
| NO | ICMP (NYHA | 0.9636 | 0.90697 | 0.89953 |
| | II or III) | | | |
| YES | CHF | 0.79459 | 0.73853 | 0.71157 |
| | (NYHA I) | | | |
| YES | CHF (NYHA | 0.88063 | 0.81459 | 0.79256 |
| | II or III) | | | |
| YES | DCMP | 0.78892 | | |
| | (NYHA I) | | | |
| YES | DCMP (NYHA | 0.84173 | 0.76323 | 0.76978 |
| | II or III) | | | |
| YES | HCMP | 0.71117 | 0.63413 | 0.61716 |
| | (NYHA I) | | | |
| YES | HCMP (NYHA | 0.80362 | 0.68967 | 0.69208 |
| | II or III) | | | |
| YES | HFrEF | 0.84977 | 0.78003 | 0.77696 |
| | (NYHA I) | | | |
| YES | HFrEF (NYHA | 0.90691 | 0.85123 | 0.78848 |
| | II or III) | | | |
| YES | ICMP | 0.88167 | 0.86139 | 0.82548 |
| | (NYHA I) | | | |
| YES | ICMP (NYHA | 0.94005 | 0.87227 | 0.85755 |
| | II or III) | | | |

1. A method for diagnosing heart failure in a subject comprising the steps of:
a) determining in a sample of a subject suspected to suffer from heart failure the amounts of a group of biomarkers, said group comprising: Cholesterylester C18:1, Cholesterylester C18:2, a Sphingomyelin C23:0, a Spingomyelin C24:0, and cysteine; and
b1) calculating a score based on the determined amounts of the biomarkers as referred to in step a), and
b2) comparing the, thus, calculated score to a reference score, whereby heart failure is to be diagnosed.

2. The method of claim 1, wherein said Spingomyelin C23:0 and/or said Spingomyelin C24:0 is/are selected from the Sphingomyelins listed in Table 1B.

3. The method of claim 1, wherein at least one further biomarker is determined being selected from the biomarkers listed in Table 2.

4. The method of claim 1, wherein said heart failure is congestive heart failure according to NYHA class I and wherein
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), TAG_Stearic acid (C18:0), Normetanephrine, and Mannose provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Normetanephrine, TAG_Stearic acid (C18:0), Noradrenaline (Norepinephrine), and Behenic acid (C22:0) provided that a correction for confounders is carried out.

5. The method of claim 1, wherein said heart failure is congestive heart failure according to NYHA class II or III and wherein
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Mannose, Normetanephrine, Lignoceric acid (C24:0), and TAG_Stearic acid (C18:0) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Mannose, Noradrenaline (Norepinephrine), Lignoceric acid (C24:0), and TAG_Stearic acid (C18:0) provided that a correction for confounders is carried out.

6. The method of claim 1, wherein said heart failure is DCMP according to NYHA class I and wherein
(i) the group of biomarkers comprises Sphingomyelin (d17:1,C24:0), Cholesterylester C18:2, Sphingomyelin (d17:1,C23:0), Cysteine, Cholesterylester C18:1, TAG_Stearic acid (C18:0), Noradrenaline (Norepinephrine), alpha-Ketoglutarate, trans-4-Hydroxyproline, and Uric acid provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), TAG_Stearic acid (C18:0), alpha-Ketoglutarate, Uric acid, and Lignoceric acid (C24:0) provided that a correction for confounders is carried out.

7. The method of claim 1, wherein said heart failure is DCMP according to NYHA class II or III and wherein
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Uric acid, Lignoceric acid (C24:0), TAG_Stearic acid (C18:0), and Noradrenaline (Norepinephrine) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Uric acid, TAG_Stearic acid (C18:0), and Mannose provided that a correction for confounders is carried out.

8. The method of claim 1, wherein said heart failure is HCMP according to NYHA class I and wherein
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, TAG_Stearic acid (C18:0), Mannose, Pyruvate, and Uric acid provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, TAG_Stearic acid (C18:0), Pyruvate, Taurine, Uric acid, and Mannose provided that a correction for confounders is carried out.

9. The method of claim 1, wherein said heart failure is HCMP according to NYHA class II or III and wherein
(i) the group of biomarkers comprises Sphingomyelin (d17:1,C24:0), Cholesterylester C18:2, Sphingomyelin (d17:1,C23:0), Cysteine, Cholesterylester C18:1, Cystine, Lactate, Lignoceric acid (C24:0), alpha-Ketoglutarate, and Mannose provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Lactate, Lignoceric acid (C24:0), TAG_Stearic acid (C18:0), Cystine, and alpha-Ketoglutarate provided that a correction for confounders is carried out.

10. The method of claim 1, wherein said heart failure is HFrEF according to NYHA class I and wherein
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), TAG_Stearic acid (C18:0), Normetanephrine, and Behenic acid (C22:0) provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Normetanephrine, TAG_Stearic acid (C18:0), and Behenic acid (C22:0) provided that a correction for confounders is carried out.

11. The method of claim 1, wherein said heart failure is HFrEF according to NYHA class II or III and wherein
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, Noradrenaline (Norepinephrine), Mannose, Glycine, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, TAG_Stearic acid (C18:0), and Behenic acid (C22:0) provided that a correction for confounders is carried out.

12. The method of claim 1, wherein said heart failure is ICMP according to NYHA class I and wherein
(i) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, alpha-Ketoglutarate, 4-Hydroxy-3-methoxyphenylglycol (HMPG), Behenic acid (C22:0), Lactate, and trans-4-Hydroxyproline provided that a correction for confounders is carried out.

13. The method of claim 1, wherein said heart failure is ICMP according to NYHA class II or III and wherein
(i) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Mannose, Behenic acid (C22:0), and Normetanephrine provided that no correction for confounders is carried out or
(ii) the group of biomarkers comprises Sphingomyelin (d17:1,C24:0), Cholesterylester C18:2, Sphingomyelin (d17:1,C23:0), Cysteine, Cholesterylester C18:1, Noradrenaline (Norepinephrine), alpha-Ketoglutarate, Behenic acid (C22:0), Mannose, and TAG_Stearic acid (C18:0) provided that a correction for confounders is carried out.

14. (canceled)

15. A device for diagnosing heart failure comprising:
a) an analysing unit comprising one at least one detector for a group of biomarkers, said group comprising Cholesterylester C18:1, Cholester-ylester C18:2, a Sphingomyelin C23:0, a Spingomyelin C24:0, and cysteine, wherein said analyzing unit is adapted for determining the amounts of the said biomarkers detected by the at least one detector, and, operatively linked thereto;
b) an evaluation unit comprising a computer comprising tangibly embedded a computer program code for carrying out a comparison of the determined amounts of the group of biomarkers and reference amounts and a data base comprising said reference amounts for the said biomarkers whereby it will be diagnosed whether a subject suffers from heart failure.
